(19) United States
(12) Patent Application Publication
(10) Pub. No.: US 2007/0015146 A1
(43) Pub. Date: Jan. 18, 2007

(54) Molecular nephrotoxicology modeling

(75) Inventors: Donna L. Mendrick, Gaithersburg, MD (US); Mark W. Porter, Gaithersburg, MD (US); Kory R. Johnson, Gaithersburg, MD (US); Arthur Castle, Gaithersburg, MD (US); Brandon Higgs, Gaithersburg, MD (US); Michael Elashoff, Gaithersburg, MD (US)

Correspondence Address: MORGAN LEWIS & BOCKIUS LLP, 1111 PENNSYLVANIA AVENUE NW, WASHINGTON, DC, 20004, US

(73) Assignee: Gene Logic, Inc.

(21) Appl. No.: 10/301,856

(22) Filed: Nov. 22, 2002

Related U.S. Application Data
(63) Continuation-in-part of application No. 10/152,319, filed on May 22, 2002.
(60) Provisional application No. 60/292,335, filed on May 22, 2001.
(60) Provisional application No. 60/297,523, filed on Jun. 13, 2001.
(60) Provisional application No. 60/298,925, filed on Jun. 19, 2001.
(60) Provisional application No. 60/303,810, filed on Jul. 10, 2001.
(60) Provisional application No. 60/303,807, filed on Jul. 10, 2001.
(60) Provisional application No. 60/303,808, filed on Jul. 10, 2001.
(60) Provisional application No. 60/315,047, filed on Aug. 28, 2001.
(60) Provisional application No. 60/324,928, filed on Sep. 27, 2001.
(60) Provisional application No. 60/330,867, filed on Nov. 1, 2001.
(60) Provisional application No. 60/330,462, filed on Oct. 22, 2001.
(60) Provisional application No. 60/331,805, filed on Nov. 21, 2001.
(60) Provisional application No. 60/336,144, filed on Dec. 6, 2001.
(60) Provisional application No. 60/340,873, filed on Dec. 19, 2001.
(60) Provisional application No. 60/357,843, filed on Feb. 21, 2002.
(60) Provisional application No. 60/357,842, filed on Feb. 21, 2002.
(60) Provisional application No. 60/357,844, filed on Feb. 21, 2002.
(60) Provisional application No. 60/364,134, filed on Mar. 15, 2002.
(60) Provisional application No. 60/370,206, filed on Apr. 8, 2002.
(60) Provisional application No. 60/370,247, filed on Apr. 8, 2002.
(60) Provisional application No. 60/370,144, filed on Apr. 8, 2002.
(60) Provisional application No. 60/371,679, filed on Apr. 12, 2002.
(60) Provisional application No. 60/372,794, filed on Apr. 17, 2002.

(51) Int. Cl. C12Q 1/68; G06F 19/00; G01N 33/48; G01N 33/50

(52) U.S. Cl. 435/6; 702/20

(57) ABSTRACT

The present invention is based on the elucidation of the global changes in gene expression and the identification of toxicity markers in kidney tissues or cells exposed to a known renal toxin. The genes may be used as toxicity markers in drug screening and toxicity assays. The invention includes a database of genes characterized by toxin-induced differential expression that is designed for use with microarrays and other solid-phase probes.

### RELATED APPLICATIONS

This application is a continuation-in-part of U.S. application Ser. No. 10/152,319, filed May 22, 2002, which claims priority under 35 U.S.C. §119(e) to U.S. Provisional Application 60/292,335, filed May 22, 2001; 60/297,523, filed Jun. 13, 2001; 60/298,925, filed Jun. 19, 2001; 60/303,810, filed Jul. 10, 2001; 60/303,807, filed Jul. 10, 2001; 60/303,808, filed Jul. 10, 2001; 60/315,047, filed Aug. 28, 2001; 60/324,928, filed Sep. 27, 2001; 60/330,867, filed Nov. 1, 2001; 60/330,462, Oct. 22, 2001; 60/331,805, filed Nov. 21, 2001; 60/336,144, filed Dec. 6, 2001; 60/340,873, filed Dec. 19, 2001; 60/357,843, filed Feb. 21, 2002; 60/357,842, filed Feb. 21, 2002; 60/357,844, filed Feb. 21, 2002; 60/364,134 filed Mar. 15, 2002; 60/370,206, filed Apr. 8, 2002; 60/370,247, filed Apr. 8, 2002; 60/370,144, filed Apr. 8, 2002; 60/371,679, filed Apr. 12, 2002; and 60/372,794, filed Apr. 17, 2002, all of which are herein incorporated by reference in their entirety.

### SEQUENCE LISTING SUBMISSION ON COMPACT DISC

The Sequence Listing submitted concurrently herewith on compact disc under 37 C.F.R. §§1.821(c) and 1.821(e) is herein incorporated by reference in its entirety. Three copies of the Sequence Listing, one on each of three compact discs are provided. Copy 1 and Copy 2 are identical. Copies 1 and 2 are also identical to the CRF. Each electronic copy of the Sequence Listing was created on Nov. 21, 2002 with a file size of 1930 KB. The file names are as follows: Copy 1—g1508901us.txt; Copy 2—g1508901us.txt; CRF—g1508901us.txt.

### BACKGROUND OF THE INVENTION

The need for methods of assessing the toxic impact of a compound, pharmaceutical agent or environmental pollutant on a cell or living organism has led to the development of procedures which utilize living organisms as biological monitors. The simplest and most convenient of these systems utilize unicellular microorganisms such as yeast and bacteria, since they are the most easily maintained and manipulated. In addition, unicellular screening systems often use easily detectable changes in phenotype to monitor the effect of test compounds on the cell. Unicellular organisms, however, are inadequate models for estimating the potential effects of many compounds on complex multicellular animals, as they do not have the ability to carry out biotransformations.

The biotransformation of chemical compounds by multicellular organisms is a significant factor in determining the overall toxicity of agents to which they are exposed. Accordingly, multicellular screening systems may be preferred or required to detect the toxic effects of compounds. The use of multicellular organisms as toxicology screening tools has been significantly hampered, however, by the lack of convenient screening mechanisms or endpoints, such as those available in yeast or bacterial systems. Additionally, previous attempts to produce toxicology prediction systems have failed to provide the necessary modeling data and statistical information to accurately predict toxic responses (e.g., WO 00/12760, WO 00/47761, WO 00/63435, WO 01/32928, and WO 01/38579).

### SUMMARY OF THE INVENTION

The present invention is based on the elucidation of the global changes in gene expression in renal tissues or cells exposed to known toxins, in particular renal toxins, as compared to unexposed tissues or cells as well as the identification of individual genes that are differentially expressed upon toxin exposure.

In various aspects, the invention includes methods of predicting at least one toxic effect of a compound, predicting the progression of a toxic effect of a compound, and predicting the renal toxicity of a compound. The invention also includes methods of identifying agents that modulate the onset or progression of a toxic response. Also provided are methods of predicting the cellular pathways that a compound modulates in a cell. The invention also includes methods of identifying agents that modulate protein activities.

In a further aspect, the invention includes probes comprising sequences that specifically hybridize to genes in Tables 1-5. In some instances, the genes are rat genes. Also included are solid supports comprising at least two of the previously mentioned probes. The invention also includes a computer system that has a database containing information identifying the expression level in a tissue or cell sample exposed to a renal toxin of a set of genes comprising at least two genes in Tables 1-5.

### DETAILED DESCRIPTION

Many biological functions are accomplished by altering the expression of various genes through transcriptional (e.g. through control of initiation, provision of RNA precursors, RNA processing, etc.) and/or translational control. For example, fundamental biological processes such as cell cycle, cell differentiation and cell death, are often characterized by the variations in the expression levels of groups of genes.

Changes in gene expression are also associated with the effects of various chemicals, drugs, toxins, pharmaceutical agents and pollutants on an organism or cell. For example, the lack of sufficient expression of functional tumor suppressor genes and/or the over expression of oncogene/protooncogenes after exposure to an agent could lead to tumorgenesis or hyperplastic growth of cells (Marshall (1991), Cell 64:313-326; Weinberg (1991), Science 254:1138-1146). Thus, changes in the expression levels of particular genes (e.g. oncogenes or tumor suppressors) may serve as signposts for the presence and progression of toxicity or other cellular responses to exposure to a particular compound.

Monitoring changes in gene expression may also provide certain advantages during drug screening and development. Often drugs are screened for the ability to interact with a major target without regard to other effects the drugs have on cells. These cellular effects may cause toxicity in the whole animal, which prevents the development and clinical use of the potential drug.

The present inventors have examined tissue from animals exposed to known renal toxins which induce detrimental kidney effects, to identify global changes in gene expression induced by these compounds. These global changes in gene expression, which can be detected by the production of expression profiles (an expression level of one or more genes), provide useful toxicity markers that can be used to monitor toxicity and/or toxicity progression by a test compound. Some of these markers may also be used to monitor or detect various disease or physiological states, disease progression, drug efficacy, and drug metabolism.

Identification of Toxicity Markers

To evaluate and identify gene expression changes that are predictive of toxicity, studies using selected compounds with well characterized toxicity have been conducted by the present inventors to catalogue altered gene expression. In the present study, indomethacin, diflunisal, colchicine, chloroform, diclofenac, menadione, sodium chromate, sodium oxalate, thioacetamide, and vancomycin were selected as known renal toxins.

Cephaloridine is an amphoteric, semi-synthetic, broad-spectrum cephalosporin derived from cephalosporin C. Cephalosporins are β-lactam-containing antibiotics which prevent bacterial growth by inhibiting polymerization of the peptidoglycan bacterial cell wall. The linear glycan chains (composed of N-acetylglucosime and N-acetylmuramic acid) are cross-linked to each other by the coupling of short chains of several amino acids, the coupling resulting from the action of a transpeptidase. It is believed that cephalosporins act by blocking the activity of the transpeptidase (Goodman & Gilman's The Pharmalogical Basis of Therapeutics 9^{th} ed., J. G. Hardman et al. Eds., McGraw Hill, New York, 1996, pp. 1074-1075, 1089-1095).

Cephaloridine is administered intramuscularly and is used to treat infections of the respiratory tract, gastrointestinal tract and urinary tract, as well as infections of soft tissue, bones and joints. Noted adverse effects include hypersensitivity reactions (such as anaphylactic shock, urticaria and bronchospasm), gastrointestinal disturbances, candidiasis, and cardiovascular and blood toxicity, in particular, toxicity to the hematopoietic system (cells responsible for the formation of red and white blood cells and platelets).

Although cephaloridine may be nephrotoxic at high dosages, it is not as harmful to the kidneys as are the aminoglycosides and polymixins. High dosages of cephaloridine may cause acute renal tubular necrosis (Cecil Textbook of Medicine, 20^{th} ed., part XII, p. 586, J. C. Bennett and F. Plum Eds., W. B. Saunders Co., Philadelphia, 1996) or drug-induced interstitial nephritis, which is accompanied by elevated IgE levels, fever, arthralgia and maculopapular rash. Renal biopsopy demonstrates edema and interstitial inflammatory lesions, mainly with lymphocytes, monocytes, eosinophils and plasma cells. Vasculitis of small vessels may develop, leading to necrotising glomerulonephritis (G. Koren, “The nephrotoxic potential of drugs and chemicals. Pharmacological basis and clinical relevance.,” Med Toxicol Adverse Drug Exp 4(1):59-72, 1989).

Cephaloridine has also been shown to reduce mitochondrial respiration and uptake of anionic succinate and carrier-mediated anionic substrate transport (Tune et al. (1990), J Pharmacol Exp Ther 252:65-69). In a study of oxidative stress and damage to kidney tissue, cephaloridine depleted reduced glutathione (GSH) and produced oxidized glutathione (GSSG) in the renal cortex. This drug also inhibited glutathione reductase and produced malondialdehyde and conjugated dienes (Tune et al. (1989), Biochem Pharmacol 38:795-802). Because cephaloridine is actively transported into the proximal renal tubule, but slowly transported across the lumenal membrane into the tubular fluid, high concentrations can accumulate and cause necrosis. Necrosis can be prevented by administering inhibitors of organic anion transport, although such treatment may be counterproductive, as cephaloridine is passed in and out of the kidney by the renal organic anion transport system (Tune et al. (1980), J Pharmacol Exp Ther 215:186-190).

Cisplatin (Pt(NH₃)₂(Cl)₂), a broad-spectrum anti-tumor agent, is commonly used to treat tumors of the testicles, ovaries, bladder, skin, head and neck, and lungs (PDR 47^{th} ed., pp. 754-757, Medical Economics Co., Inc., Montvale, N.J., 1993; Goodman & Gilman's The Pharmalogical Basis of Therapeutics 9^{th} ed., pp. 1269-1271, J. G. Hardman et al. Eds., McGraw Hill, New York, 1996). Cisplatin diffuses into cells and functions mainly by alkylating the N⁷ of guanine, a highly reactive site, causing interstrand and intrastrand crosslinks in the DNA that are lethal to cells. The drug is not sensitive to the cell cycle, although its effects are most pronounced in S phase.

Because the drug is cleared from the body mainly by the kidneys, the most frequent adverse effect of cisplatin usage is nephrotoxicity, the severity of which increases with increasing dosage and treatment terms. Other adverse effects include renal tubule damage, myelosuppression (reduced numbers of circulating platelets, leukocytes and erythrocytes), nausea and vomiting, ototoxicity, serum electrolyte disturbances (decreased concentrations of magnesium, calcium, sodium, potassium and phosphate, probably resulting from renal tubule damage), increased serum concentrations of urea and creatinine, and peripheral neuropathies.

In one study on rats (Nonclercq et al. (1989), Exp Mol Pathol 51:123-140) administration of cisplatin or carboplatin induced renal injury, carboplatin causing less damage than cisplatin. The most prominent injury was to the straight portion of proximal renal tubule.

In another rat study (Goldstein et al. (1981), Toxicol Appl Pharmacol 60:163-175) animals injected with cisplatin displayed decreased food intake as drug dosage increased. On day 2, the high-dose groups (10-15 mg/kg) exhibited a six or seven-fold elevation in BUN. On day 4, BUN elevation was noted in the 5 mg/kg group. An increase in urine volume was observed beginning on days 3-4, along with decreased urine osmolality in the low-dose groups (2.5 or 5 mg/kg). Another experiment on rats (Agarwal et al. (1995), Kidney Int 48:1298-1307) showed that cisplatin treatment produced elevations in serum creatinine levels, which began on day 3 and progressed for the duration of the study.

PAN (C₂₂H₂₉N₇O₅), an antibiotic produced by Streptomyces alboniger, inhibits protein synthesis and is commonly used experimentally on rats to mimic human minimal change disease. One study showed that PAN-injected rats demonstrated an increase in levels of serum non-esterified fatty acids, while the serum albumin concentration was negatively affected (Sasaki et al. (1999), Adv Exp Med Biol 467:341-346).

In another rat study, an adenosine deaminase inhibitor prevented PAN nephrotoxicity, indicating that PAN toxicity is linked to adenosine metabolism (Nosaka et al. (1997), Free Radic Biol Med 22:597-605). Another group showed that PAN, when administered to rats, led to proteinuria, a condition associated with abnormal amounts of protein in the urine, and renal damage, e.g. blebbing of glomerular epithelial cells, focal separation of cells from the glomerular basement membrane, and fusion of podocytes (Olson et al. (1981), Lab Invest 44:271-279). In another study on rats, administration of PAN induced glomerular epithelial cell apoptosis in a dose- and time-dependent manner (Sanwal et al. (2001), Exp Mol Pathol 70:54-64).

One study with PAN-injected rats (Koukouritaki et al. (1998), J Investig Med 46: 284-289) examined the changes in the expression of the proteins paxillin, focal adhesion kinase, and Rho, all of which regulate cell adhesion to the extracellular matrix. Paxillin levels increased steadily, peaked at day 9 after PAN injection, and then remained elevated even after proteinuria resolved. There was no observed change in expression of either focal adhesion kinase or Rho.

BEA, (C₂H₆BrN.HBr), is commonly used experimentally on rats to induce papillary necrosis and renal cortex damage, which is similar to human analgesic nephropathy. BEA-induced papillary necrosis in rats eventually leads to the onset of focal glomerular sclerosis and nephrotic proteinuria (Garber et al. (1999), Am J Kidney Dis 33: 1033-1039). Even at low doses (50 mg/kg), BEA can induce an apex limited renal papillary necrosis (Bach et al. (1983), Toxicol Appl Pharmacol 69:333-344). In male Wistar rats, BEA administered at 100 mg/kg was shown to cause renal papillary necrosis within 24 hours (Bach et al. (1991), Food Chem Toxicol 29:211-219). Additionally, Bach et al. showed that there was an increase in urinary triglycerides, and lipid deposits were seen by Oil Red 0 lipid staining in the cells of the collecting ducts and hyperplastic urothelia adjacent to the necrosed region.

It has also been shown that succinate and citrate concentrations are significantly lower in the urine of BEA-treated rats (Holmes et al. (1995), Arch Toxicol 70:89-95). Moreover, BEA treatment induced glutaric and adipic aciduria, which is symptomatic of an enzyme deficiency in the acyl CoA dehydrogenases. The same study examined urinary taurine levels in desert mice, and in BEA-treated desert mice there was an increase in the urinary taurine level which is indicative of liver toxicity.

Another study on BEA-treated rats showed that there was an increase in the concentrations of creatine in the renal papilla and glutaric acid in the liver, renal cortex, and renal medulla as soon as 6 hours post-treatment (Garrod et al. (2001), Magn Reson Med 45: 781-790).

Discovered and purified in the early 1960's, gentamicin is a broad-spectrum aminoglycoside antibiotic that is cidal to aerobic gram-negative bacteria and commonly used to treat infections, e.g., those of the urinary tract, lungs and meninges. As is typical for an aminoglycoside, the compound is made of two amino sugar rings linked to a central aminocyclitol ring by glycosidic bonds. Aminoglycosides are absorbed poorly with oral administration, but are excreted rapidly by the kidneys. As a result, kidney toxicity is the main adverse effect, although ototoxicity and neuromuscular blockade can also occur. Gentamicin acts by interfering with bacterial protein synthesis. This compound is more potent than most other antibacterial inhibitors of protein synthesis, which are merely bacteriostatic, and its effects on the body are, likewise, more severe (Goodman & Gilman's The Pharmalogical Basis of Therapeutics 9^{th} ed., pp. 1103-1115, J. G. Hardman et al. Eds., McGraw Hill, New York, 1996).

Aminoglycosides work rapidly, and the rate of bacterial killing is concentration-dependent. Residual bactericidal activity remains after serum concentration has fallen below the minimum inhibitory concentration (MIC), with a duration that is also dosage/concentration-dependent. The residual activity allows for once-a-day administration in some patients. These drugs diffuse into bacterial cells through porin channels in the outer membrane and are then transported across the cytoplasmic membrane via a membrane potential that is negative on the inside (Goodman & Gilman, supra).

Kidney damage, which can develop into renal failure, is due to the attack of gentamicin on the proximal convoluted tubule, particularly in the S1 and S2 segments. The necrosis, however, is often patchy and focal (Shanley et al. (1990), Ren Fail 12:83-87). A rat study by Shanley et al. showed that superficial nephrons are more susceptible to necrosis than juxtamedullary nephrons, although the initial segment of the superficial nephrons is remarkably resistant to necrosis.

Reported enzymatic changes upon gentamicin treatment are increased activities of N-acetyl-beta-D-glucosaminidase and alkaline phosphatase and decreased activities of sphingomyelinase, cathepsin B, Na⁺/K⁺-ATPase, lactate dehydrogenase and NADPH cytochrome C reductase, along with decreased protein synthesis and alpha-methylglucose transport (Monteil et al. (1993), Ren Fail 15:475-483). An increase in gamma-glutamyl transpeptidase activity in urine has also been reported (Kocaoglu et al. (1994), Arch Immunol Ther Exp (Warsz) 42:125-127), and the quantification of this enzyme in urine is a useful marker for monitoring gentamicin toxicity.

One source of renal pathology resulting from gentamicin treatment is the generation of reactive oxygen metabolites. Gentamicin has been shown, both in vitro and in vivo, to be capable of enhancing the production of reactive oxygen species. Iron, a necessary co-factor that catalyzes free-radical formation, is supplied by cytochrome P450 (Baliga et al. (1999), Drug Metab Rev 31:971-997).

A gene delivery experiment in rats, in which the human kallikrein gene was cloned into an adenovirus vector and the construct then co-administered with a gentamicin preparation, showed that kallikrein can protect against gentamicin-induced nephrotoxicity. Significantly increased renal blood flow, glomerular filtration rates and urine flow were observed, along with decreased renal tubular damage, cellular necrosis and lumenal protein casts. Kallikrein gene delivery also caused a decrease in blood urea nitrogen levels and increases in urinary kinin and nitrite/nitrate levels. This study provides evidence that the tissue kallikrein-kinin system may be a key pathway that is perturbed during the induction of nephrotoxicity by gentamicin (Murakami et al. (1998), Kidney Int 53:1305-1313).

Ifosfamide, an alkylating agent, is commonly used in chemotherapy to treat testicular, cervical, and lung cancer. Ifosfamide is slowly activated in the liver by hydroxylation, forming the triazene derivative 5-(3,3-dimethyl-1-triazeno)-imidazole-4-carboxamide (DTIC) (Goodman & Gilman's The Pharmacological Basis of Therapeutics 9^{th}ed., p. 1235, J. G. Hardman et al., Eds., McGraw Hill, New York, 1996). Cytochrome P450 activates DTIC via an N-demethylation reaction yielding an alkylating moiety, diazomethane. The active metabolites are then able to cross-link DNA causing growth arrest and cell death. Though ifosfamide is therapeutically useful, it is also associated with nephrotoxicity, urotoxicity, and central neurotoxicity.

Mesna, another therapeutic, is often administered concomitantly to prevent kidney and bladder problems from arising (Brock and Pohl (1986), IARC Sci Publ 78:269-279). However, there are documented cases in which tubular toxicity occurred and elevated urinary levels of alanine aminopeptidase and N-acetyl-beta-D-glucosaminidase were found in patients even though mesna was administered alongside ifosfamide (Goren et al. (1987), Cancer Treat Rep 71:127-130).

One study examined 42 patients that had been administered ifosfamide to treat advanced soft-tissue sarcoma (Stuart-Harris et al. (1983), Cancer Chemother Pharmacol 11:69-72). The ifosfamide dosage varied from 5.0 g/m² to 8.0 g/m², and all of the patients were given mesna to counteract the negative effects of ifosfamide. Even so, nausea and vomiting were common to all of the patients. Out of the 42 patients, seven developed nephrotoxicity, and two of the cases progressed to fatal renal failure.

In another clinical study, renal tubular function was monitored in 18 neuroblastoma patients (Caron et al. (1992), Med Pediatr Oncol 20:42-47). Tubular toxicity occurred in at least 12 of the patients, and seven of those patients eventually developed Debre-de Toni-Fanconi syndrome, although in 3 cases the syndrome was reversible.

Fanconi syndrome is a disorder marked by dysfunction of the proximal tubules of the kidney. It is associated with aminoaciduria, renal glycosuria, and hyperphosphaturia. Ifosfamide is often used experimentally on rats to induce Fanconi syndrome. In one study, rats that were administered 80 mg/kg of ifosfamide had significantly lower body weight and hematocrit than control rats (Springate and Van Liew (1995), J Appl Toxicol 15:399-402). Additionally, the rats had low-grade glucosuria, proteinuria, and phosphaturia. In a mouse study, ifosfamide induced elevated serum creatinine and urea levels and decreased the clearance rate of creatinine (Badary (1999), J Ethnopharmacol 67:135-142).

Cyclophosphamide, a nitrogen mustard and alkylating agent, is highly toxic to dividing cells and is commonly used in chemotherapy to treat malignant lymphomas, such as non-Hodgkin's lymphomas and Burkitt's lymphoma, multiple myeloma, leukemias, neuroblastomas, ovarian adenocarcinomas and retinoblastomas, as well as breast and lung cancer (Goodman & Gilman's The Pharmacological Basis of Therapeutics 9^{th} ed., pp. 1234, 1237-1239, J. G. Hardman et al., eds., McGraw Hill, New York, 1996; Physicians Desk Reference, 47^{th} ed., pp. 744-745, Medical Economics Co., Inc., Montvale, N.J., 1993). Additionally, cyclophosphamide is used as an immunosuppressive agent in bone marrow transplantation and following organ transplantation. Although cyclophosphamide is therapeutically useful against certain types of cancer, it is also associated with cardiotoxicity, nephrotoxicity (including renal tubular necrosis), hemorrhagic cystitis, myelosuppression, hepatotoxicity, impairment of male and female reproductive systems, interstitial pneumonitis and central nervous system toxicity.

Once in the liver, cyclophosphamide is hydroxylated by the cytochrome P450 mixed function oxidase system, producing the active metabolites phosphoramide mustard and acrolein, which cross-link DNA and cause growth arrest and cell death. These metabolites, however, are highly toxic and cause adverse effects in the other organs into which they are transported, such as the kidneys. Acrolein is removed from the kidneys by secretion into the urine, resulting in cystitis (inflammation of the bladder), often hemorrhagic cystitis.

In the kidney, cyclophosphamide induces necrosis of the renal distal tubule. Cyclophosphamide, which is structurally similar to the anti-cancer drug ifosfamide, does not induce damage to the renal proximal tubule nor does it induce Debre-de Toni-Fanconi syndrome (Rossi et al. (1997), Nephrol Dial Transplant 12:1091-1092).

One clinical trial of patients being treated with cyclophosphamide showed that renal damage from the drug leads to a reduced biotransformation rate and low renal clearance of the drug, resulting in a build-up of toxic alkylating metabolic products (Wagner et al. (1980), Arzneimittelforschung 30:1588-1592).

In a study of patients suffering from malignant lymphomas and mammary carcinomas, a direct relationship was found between the dose of cyclophosphamide used in treatment and the concentration of alkylating metabolites in the patients' urine. The upper limit of the dose was determined by the nature and degree of the toxic side effects, rather than by the rate at which the drug could be metabolized (Saul et al (1979), J Cancer Res Clin Oncol 94:277-286). It is the acrolein itself that is toxic, not the alkylating activity of cyclophosphamide (Brock et al. (1979), Arzneimittelforschung 29:659-661). A study on rats also showed that acrolein from the kidneys can produce hemorrhagic cystitis and that the acrolein concentration is directly related to the frequency and severity of the cystitis (Chijiwa et al. (1983), Cancer Res 43:5205-5209).

Carboplatin, a platinum coordination complex, is commonly used in chemotherapy as an anti-tumor agent. As a chemotherapeutic agent, carboplatin acts similarly to cisplatin. Carboplatin enters the cell by diffusion where it is activated by hydrolysis (Goodman & Gilman's The Pharmacological Basis of Therapeutics 9^{th} ed., p. 1270-1271, J. G. Hardman et al. Eds., McGraw Hill, New York 1996). Once activated, the platinum complexes are able to react with DNA causing cross-linking to occur. One of the differences between carboplatin and cisplatin is that carboplatin is better tolerated clinically. Some of the side-effects associated with cisplatin, such as nausea, neurotoxicity, and nephrotoxicity, are seen at a lesser degree in patients administered carboplatin. Some other side-effects are hypomagnesaemia and hypokalaemia (Kintzel (2001), Drug Saf 24:19-38).

In one study on male Wistar rats, carboplatin was administered at a dosage of 65 mg/kg (Wolfgang et al. (1994), Fundam Appl Toxicol 22:73-79). After treatment with carboplatin, CGT excretion was increased approximately two-fold.

Another study compared cisplatin and carboplatin when given in combination with vindesine and mitomycin C (Jelic et al. (2001) Lung Cancer 34:1-13). The study showed that carboplatin administered with vindesine and mitomycin C was advantageous in terms of overall survival, although the regimen was more hematologically toxic than when cisplatin was given.

AY-25329, is a phenothiazine that has been shown to be mildly hepatotoxic and to induce nephrosis. Its structure is shown below.

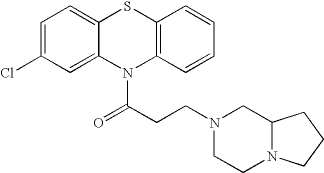

Phenothiazines are a class of psychoactive drugs. They have been used to treat schizophrenia, paranoia, mania, hyperactivity in children, some forms of senility, and anxiety (http://www.encyclopedia.com/articlesnew/36591.html). Some side effects associated with prolonged use of the drugs are reduced blood pressure, Parkinsonism, reduction of motor activity, and visual impairment.

Chlorpromazine (Thorazine or Largactil) is an aliphatic phenothiazine and is widely used for treating schizophrenia and manic depression. Prolactin secretion is increased while taking chlorpromazine, and galactorrhea and gynecomastia have both been associated with the drug (http://www.mentalhealth.com/drug/p30-c01.html). Trifluoperazine is another prescribed phenothiazine. It is used to treat anxiety, to prevent nausea and vomiting, and to manage psychotic disorders (http://www.mentalhealth.com/drug/p30-s04.html). Negative side-effects that have been associated with the drug are liver damage, bone marrow depression, and Parkinsonism.

Acyclovir (9-[(2-hydroxyethyl)methyl] guanine, Zovirax®), an anti-viral guanosine analogue, is used to treat herpes simplex virus (HSV), varicella zoster virus (VZV) and Epstein-Barr virus (EBV) infections. It is transported into cells by the nucleoside transporter that imports guanine, and acyclovir is phosphorylated by virally encoded thymidine kinase (TK). Other kinases convert acyclovir to its activated di- and triphosphate forms, which prevent the polymerization of viral DNA. Acyclovir triphosphate competes with dGTP for the viral polymerase, and acyclovir is preferentially incorporated, but as a monophosphate. As a result, chain elongation ceases (Fields Virology 3d ed., Fields et al., eds., pp. 436-440, Lippincott-Raven Publishers, Philadelphia, 1996; Cecil Textbook of Medicine, 20^{th} ed., part XII, p. 1742, J. C. Bennett and F. Plum Eds., W. B. Saunders Co., Philadelphia, 1996).

The pharmacokinetics of acyclovir show that it has a useful half-life of about three hours and that most of it is excreted in the urine largely unchanged (Brigden et al. (1985), Scand J Infect Dis Suppl 47:33-39). Not surprisingly, the most frequent adverse effect of acyclovir treatment is damage to various parts of the kidney, particularly the renal tubules. Crystalluria, or the precipitation of crystals (in this case, crystals of acyclovir), in the lumina of the renal tubules can occur (Fogazzi (1996), Nephrol Dial Transplant 11:379-387). If the drug crystallizes in the renal collecting tubules, obstructive nephropathy and tubular necrosis can result (Richardson (2000), Vet Hum Toxicol 42:370-371). Tissues from biopsies of affected patients showed dilation of the proximal and distal renal tubules, with loss of the brush border, flattening of the lining cells and focal nuclear loss (Becker et al. (1993), Am J Kidney Dis 22:611-615).

Citrinin, a mycotoxin produced by the fungus Penicillium citrinum, is a natural contaminant of foods and feeds (Bondy and Armstrong (1998) Cell Biol. Toxicol. 14:323-332). It is known that mycotoxins can have negative effects on the immune system, however citrinin-treated animals have been shown to stimulate responses against antigens (Sharma (1993) J Dairy Sci. 76:892-897). Citrinin is a known nephrotoxin, and in birds such as chickens, ducklings, and turkeys, it causes diarrhea, increased food consumption and reduced weight gain due to kidney degeneration (Mehdi et al. (1981) Food Cosmet. Toxicol. 19:723-733; Mehdi et al. (1984) Vet. Pathol. 21:216-223). In the turkey and duckling study, both species exhibited nephrosis with the occurrence of hepatic and lymphoid lesions (Mehdi et al., 1984).

In one study, citrinin was administered to rabbits as a single oral dose of either 120 or 67 mg/kg (Hanika et al. (1986) Vet. Pathol. 23:245-253). Rabbits treated with citrinin exhibited renal alterations such as condensed and distorted mitochondria, distended intercellular spaces of the medullary and straight cortical distal tubules, and disorganization of interdigitating processes. In another rabbit study, citrinin-administered rabbits displayed azotaemia and metabolic acidosis (Hanika et al. (1984) Food Chem. Toxicol. 22:999-1008). Renal failure was indicated by decreased creatinine clearance and increased blood urea nitrogen and serum-creatinine levels.

In the past, mercury was an important component of pharmaceuticals, particularly of antiseptics, antibacterials, skin ointments, diuretics and laxatives. Although, mercury has been largely replaced by more effective, more specific and safer compounds, making drug-induced mercury poisoning rare, it is still widely used in industry. Poisoning from occupational exposure and environmental pollution, such as mercury release into public water supplies, remains a concern as wildlife, domestic animals and humans are affected.

Because of their lipid solubility and ability to cross the blood-brain barrier, the most dangerous form of mercury is the organomercurials, the most common of which is methylmercury, a fungicide used for disinfecting crop seeds. In a number of countries, incidents involving large-scale illness and death from mercury poisoning have been reported when mercury-contaminated seeds were planted and the crops harvested and consumed. A second source of organic mercury poisoning results from industrial chemicals containing inorganic mercury, such as mercury catalysts, which form methylmercury as a reaction product. If this waste product is released into reservoirs, lakes, rivers or bays, the surrounding population can become sick or die, particularly those who eat local fish.

The inorganic salt mercuric chloride, HgCl₂, as well as other mercuric salts, are more irritating and more toxic than the mercurous forms. Mercuric chloride is used today in industry, for the manufacture of bleach, electronics, plastics, fungicides and dental amalgams. The main source of human exposure is industrial dumping into rivers (Goodman & Gilman's: The Pharmacological Basis of Therapeutics (9th ed.), pp. 1654-1659, McGraw-Hill, New York, 1996).

When inorganic mercury salts are ingested, about 10% of the mercuric ions are absorbed by the gastrointenstinal tract, and a considerable portion of the Hg²⁺ can remain bound to the mucosal surfaces. The highest concentration of Hg²⁺ is found in the kidneys, as it is retained there longer than in other tissues. Consequently, the kidneys are the organ most adversely affected by inorganic mercury poisoning. The proximal tubules are the major site of damage, where tubular necrosis results. The mercury affects primarily the S2 and S3 portions of the proximal tubules, but, at high levels of mercury exposure, the S1 and distal portions of the tubules are also damaged. These regions of the nephrons are affected because they contain enzymes (such as gamma-glutamyltranspeptidase) and transport proteins (such as the basolateral organic anion transport system) involved in mercury uptake (Diamond et al. (1998), Toxicol Pathol 26:92-103).

Urinary markers of mercury toxicity which can be detected in NMR spectra include elevated levels of lactate, acetate and taurine and decreased levels of hippurate (Holmes et al. (2000), Chem Res Toxicol 13:471-478). Known changes in gene expression in kidneys exposed to Hg²⁺ include up-regulation of the heat-shock protein hsp72 and of the glucose-regulated protein grp94. The degree of tissue necrosis and level of expression of these proteins is proportional to both the dose of mercury (Hg²⁺) and the length of the exposure time to mercury (Hg²⁺), with hsp72 accumulating in the renal cortex and grp94 accumulating in the renal medulla (Goering et al (2000), Toxicol Sci 53:447-457).

Indomethacin is a non-steroidal antiinflammatory, antipyretic and analgesic drug commonly used to treat diseases such as rheumatoid arthritis, osteoarthritis, ankylosing spondylitis and gout. This drug acts as a potent inhibitor of prostaglandin synthesis; it inhibits the cyclooxygenase enzyme necessary for the conversion of arachidonic acid to prostaglandins (PDR 47th ed., Medical Economics Co., Inc., Montvale, N.J., 1993; Goodman & Gilman's The Pharmalogical Basis of Therapeutics 9th ed., J. G. Hardman et al., Eds., McGraw Hill, New York, 1996, pp. 1074-1075, 1089-1095; Cecil Textbook of Medicine, 20th ed., part XII, pp. 772-773, 805-808, J. C. Bennett and F. Plum Eds., W. B. Saunders Co., Philadelphia, 1996).

The most frequent adverse effects of indomethacin treatment are gastrointestinal disturbances, e.g., bleeding, ulcers and perforations, although renal toxicity can also result, particularly after long-term administration. In rats, hemorrhage and necrosis have been observed in the renal papillae and formix, as well as damage to the thick ascending limbs (mTALs), and interstitial nephritis with hematuria, proteinuria and nephrotic syndrome have been reported in humans. Patients suffering from renal dysfunction risk developing a reduction in renal blood flow and urinary outflow, because renal prostaglandins play an important role in renal perfusion and glomerular filtration (Heyman et al. (1997), Kidney Int 51: 653-663).

Diflunisal, a non-steroidal anti-inflammatory drug (NSAID), is a difluorophenyl derivative of salicylic acid (Goodman & Gilman's The Pharmacological Basis of Therapeutics 9^{th} ed., p. 631, J. G. Hardman et al., Eds., McGraw Hill, New York, 1996). It is most frequently used in the treatment of osteoarthritis and musculoskeletal strains. NSAIDs have analgesic, antipyretic and anti-inflammatory actions, however hepatotoxicity is known to be an adverse side effect of NSAID treatment (Masubuchi et al. (1998) J. Pharmacol. Exp. Ther. 287:208-213). Diflunisal has been shown to be less toxic than other NSAIDs, nevertheless over long periods of dosage it can lead to deleterious effects on platelet or kidney function (Bergamo et al. (1989) Am. J. Nephrol. 9:460-463). Other side effects that have been associated with diflunisal treatment are diarrhea, dizziness, drowsiness, gas or heartburn, headache, nausea, vomiting, and insomnia (http://arthritisinsight.com/medical/meds/dolobid.html).

Masubuchi et al. compared the hepatotoxicity of 18 acidic NSAIDs. In the study, diflunisal (administered at a concentration of 500 μM) was shown to increase LDH leakage in rat hepatocytes, a marker for cell injury, when compared to the control sample. In addition, treatment with diflunisal led to decreased intracellular ATP concentrations.

One study compared the effects of diflunisal and ibuprofen when given to patients over a two week period (Muncie and Nasrallah (1989) Clin. Ther. 11:539-544). In both the ibuprofen and the diflunisal group, two patients complained of abdominal cramping. The study indicated that even during short-term usage some gastrointestinal effects may occur. The toxic dose used in this study was chosen as one that did not induce significant gastric ulceration in rats. The group of rats given the high dosage of diflunisal had increased concentrations of creatinine which is consistent with renal injury, although dehydration may also cause increases in creatinine concentration.

Cidofovir (Vistide®) is an antiviral cytosine analog used in the treatment of viral infections such as herpesvirus, adenovirus, papillomavirus, poxvirus and hepadnavirus (Goodman & Gilman's The Pharmacological Basis of Therapeutics 9^{th} ed., p. 1216, J. G. Hardman et al., Eds., McGraw Hill, New York, 1996). It is also useful for the treatment of cytomegalovirus (CMV) infection, which is a type of herpesvirus.

Some mild side effects seen in patients receiving cidofovir are nausea, vomiting, and fever. The most serious reported side effect of the drug is kidney toxicity (http://tthivclinic.com/cido.html). In response to the threat of nephrotoxicity, it is necessary for patients receiving cidofovir to have their kidneys checked before treatment, and the patients must be monitored during treatment for early symptoms of kidney problems. In addition, cidofovir is given with fluids to help reduce the risk of kidney toxicity (http://www.aidsinfonyc.org/network/simple/cido.html). Probenecid, a drug that helps protect the kidneys, is normally administered concomitantly (Lalezari and Kuppermann (1997) J. Acquir. Immune Defic. Syndr. Hum. Retrovirol. 14:S27-31).

One study compared the safety and efficacy of cidofovir in the treatment of CMV (Lalezari et al. (1998) J. Acquir. Immune Defic. Syndr. Hum. Retrovirol. 17:339-344). Approximately 40% of the patients exhibited dose-dependent asymptomatic proteinuria and 25% of the patients had elevated serum creatinine levels.

Pamidronate (Aredia®) is a bisphosphonate drug that is clinically used to inhibit bone resorption and make bones more stable. It is used to treat hypercalcemia (too much calcium in the blood) that occurs with some types of cancer. Typically administered by intravenous injection, pamidronate is frequently used in patients with breast cancer or multiple myeloma whose disease has spread to the bones. Some side effects related to pamidronate treatment are abdominal cramps, chills, confusion, fever, muscle spasms, nausea, muscle stiffness, and swelling at the injection site (http://www.nursing.uiowa.edu/sites/PedsPain/Adjuvants/PAMIDRnt.html). Patients with kidney problems may be prohibited from using pamidronate as it is excreted through the kidneys.

In one study, rats and mice were given varying doses of labeled pamidronate (Cal and Daley-Yates (1990) Toxicology 65:179-197). Pamidronate treatment led to significant weight loss and a decrease in creatinine clearance. Morphological studies showed a loss of brush border membranes and the presence of focal proximal tubular necrosis.

Another study compared the tolerability of different treatments for hypercalcemia of malignancy by reviewing articles published between 1979 and 1998 (Zojer et al. (1999) Drug Saf 21:389-406). The authors found that elevated serum creatinine level, nausea, and fever were reported following treatment with bisphosphonates such as pamidronate.

Markowitz et al. (2001, J. Am. Soc. Nephrol. 12:1164-1172) tried to determine whether there was a correlation between pamidronate treatment and collapsing focal segmental glomerulosclerosis (FSGS). The authors examined the histories of seven patients who had developed collapsing FSGS, and they found that the only drug treatment in common was the administration of pamidronate. When given at the recommended dose of 90 mg per month, renal toxicity was rare. However, when pamidronate was given at higher doses nephrotoxicity occurred.

Lithium, an alkali metal, is the main pharmacological treatment for bipolar disorders. It is typically given as a salt, such as lithium carbonate or lithium citrate. Some common side effects of lithium treatment are an increase in urination, increase in drinking, dry mouth, weight gain, fine tremor, and fatigue. Some more serious side effects related to lithium treatment are blurred vision, mental confusion, seizures, vomiting, diarrhea, muscle weakness, drowsiness, and coarse tremor (Goodman & Gilman's The Pharmacological Basis of Therapeutics 9^{th} ed., p. 448, J. G. Hardman et al., Eds., McGraw Hill, New York, 1996).

Since lithium is often used on a maintenance basis for a lifelong period, numerous studies have been performed to try and elucidate the effects of lithium on the kidney. One group administered lithium in daily doses within the human therapeutic range to male Wistar rats (Kling et al (1984) Lab Invest 50:526-535). Rats that were given lithium developed marked polyuria within three weeks of the initial dosing. The rats displayed elevated free water clearance and vasopressin-resistant diabetes insipidus. The cortical collecting tubules displayed morphological changes, e.g. dilation of the tubules, bulging cells lining the tubules, enlarged nuclei, following lithium treatment.

Another study examined a human population that had been given lithium for the treatment of bipolar disorder (Markowitz et al. (2000) J. Am. Soc. Nephrol. 11: 1439-1448). The patients had a mean age of 42.5 years and had been undergoing lithium treatment from 2 to 25 years (mean of 13.6 years). Approximately one fourth of the patients had nephrotic proteinuria, almost 90% of them had nephrogenic diabetes insipidus (NDI), and renal biopsies revealed a chronic tubulointerstitial nephropathy in all of the patients. Following cessation of lithium treatment, seven of the patients proceeded to end-stage renal disease.

Even though nephrotoxicity is a known side effect of lithium treatment, some studies have indicated that in actuality it is not all that common (Johnson (1998) Neuropsychopharmacology 19:200-205). One study showed that the NDI-like effect in lithium treatment was easily overcome by increasing the levels of arginine vasopressin (AVP) (Carney et al. (1996) Kidney Int 50:377-383). Other studies have suggested that patients with psychiatric disorders display certain defects in renal function without undergoing lithium treatment (Gitlin (1999) Drug Saf 20:231-243).

Hydralazine, an antihypertensive drug, causes relaxation of arteriolar smooth muscle. Such vasodilation is linked to vigorous stimulation of the sympathetic nervous system, which in turn leads to increased heart rate and contractility, increased plasma renin activity, and fluid retention (Goodman & Gilman's The Pharmacological Basis of Therapeutics 9^{th} ed., p. 794, J. G. Hardman et al., Eds., McGraw Hill, New York, 1996). The increased renin activity leads to an increase in angiotensin II, which in turn causes stimulation of aldosterone and sodium reabsorption.

Hydralazine is used for the treatment of high blood pressure (hypertension) and for the treatment of pregnant women suffering from high blood pressure (pre-eclampsia or eclampsia). Some common side effects associated with hydralazine use are diarrhea, rapid heartbeat, headache, decreased appetite, and nausea. Hydralazine is often used concomitantly with drugs that inhibit sympathetic activity to combat the mild pulmonary hypertension that can be associated with hydralazine usage.

In one hydralazine study, rats were fed hydralazine and mineral metabolism was monitored (Peters et al. (1988) Toxicol Lett 41:193-202). Manganese and zinc concentrations were not effected by hydralazine treatment, however tissue iron concentrations were decreased and kidney copper concentrations were increased compared to control groups.

Another study compared the effects of hydrazine, phenelzine, and hydralazine treatment on rats (Runge-Morris et al. (1996) Drug Metab Dispos 24:734-737). Hydralazine caused an increase in renal GST-alpha subunit expression, although unlike hydrazine and phenelzine it did not alter renal cytochrome P4502E1 expression.

Colchicine, an alkoloid of Colchicum autumale, is an antiinflammatory agent used in the treatment of gouty arthritis (Goodman & Gilman's The Pharmacological Basis of Therapeutics 9^{th} ed., p. 647, J. G. Hardman et al., Eds., McGraw Hill, New York, 1996).

An antimitotic agent, colchicine binds to tubulin which leads to depolymerization and disappearance of the fibrillar microtubules in granulocytes and other motile cells. In doing so, the migration of granulocytes into the inflamed area is inhibited. Through a series of events, the inflammatory response is blocked.

Some common, mild side effects associated with colchicine treatment are loss of appetite and hair loss. More severe side effects that warrant cessation of treatment are nausea, vomiting, diarrhea, and abdominal pain. Colchicine overdose can induce multiorgan failure with a high incidence of mortality. In this setting, renal failure is multifactorial and related to prolonged hypotension, hypoxemia, sepsis, and rhabdomyolysis. In rats, less dramatic doses have been shown to inhibit the secretion of many endogenous proteins such as insulin and parathyroid hormone.

One study investigated the effects of colchicine on microtubule polymerization status and post-translational modifications of tubulin in rat seminiferous tubules (Correa and Miller (2001) Biol Reprod 64:1644-1652). Colchicine caused extensive microtubule depolymerization, and total tubulin levels decreased twofold after colchicine treatment. The authors also found that colchicine treatment led to a decrease in tyrosination of the microtubule pool of tubulin which was associated with depolymerization of microtubules.

Sulfadiazine, a sulfonamide, is an antimicrobial agent. It is commonly used concomitantly with pyrimethamine to treat toxoplasmosis, an infection of the brain, in patient suffering from AIDS. These drugs are able to cross the blood-brain barrier and are used at relatively high doses. In addition, sulfadiazine has been shown to be effective at preventing certain types of meningococcal diseases and in treating urinary tract infections.

Sulfonamides in general are structural analogs of para-aminobenzoic acid (PABA). Because they are competitive antagonists of PABA, sulfonamides are effective against bacteria that are required to utilize PABA for the synthesis of folic acid (Goodman & Gilman's The Pharmacological Basis of Therapeutics 9^{th} ed., p. 1058-1060, J. G. Hardman et al., Eds., McGraw Hill, New York, 1996).

The main side effects associated with sulfadiazine treatment are fever and skin rashes. Decreases in white blood cells, red blood cells, and platelets, nausea, vomiting, and diarrhea are some other side effects that may result from sulfadiazine treatment. The most troublesome problem with this drug for HIV/AIDS patients is kidney toxicity. These patients tend to use these drugs for extended periods of time, which puts a constant strain on the kidneys. In addition, kidney stones tend to form in the bladder and ureter thereby blocking the flow of urine. Kidney damage may result, and if left untreated kidney failure may occur. Therefore, patients being treated with sulfadiazine are instructed to increase their fluid intake in order to prevent crystal formation in the kidneys.

One case study examined four HIV-positive patients who had been given sulfadiazine to treat toxoplasmosis (Crespo et al. (2000) Clin Nephrol 54:68-72). All four of the patients, one of whom was a previously healthy person, developed oliguria, abdominal pain, renal failure, and displayed multiple radiolucent renal calculi in echography. Following extensive hydration and alcalinization, the renal function of the patients returned to normal.

Adriamycin, known generically as doxorubicin, is an anthracycline antibiotic produced by the fungus Streptomyces peucetius. It is an anti-tumor drug used in the treatment of breast, ovarian, bladder, and lung cancers as well as non-Hodgkin's lymphoma, Hodgkin's disease and sarcoma (Goodman & Gilman's The Pharmacological Basis of Therapeutics 9^{th} ed., p. 1264-1265, J. G. Hardman et al., Eds., McGraw Hill, New York, 1996).

Adriamycin has tetracycline ring structures with the sugar daunosamine attached by glycosidic linkage. It is able to intercalate with DNA, it affects DNA and RNA synthesis, and it can interact with cell membranes and alter their functions. Typically the drug is cell-cycle specific for the S phase of cell division. By binding to the cancer cells' DNA and blocking topoisomerase II, cancer cells are unable to divide and grow.

Some common side effects associated with adriamycin treatment are fatigue, a drop in white blood cell, red blood cell, or platelet count, hair loss, skin discoloration, and watery eyes (www.cancerhelp.org.uk/help/default.asp?page=4025). More serious side effects include myocardial toxicity, ulceration and necrosis of the colon, and development of a second cancer.

Because of its utility in fighting cancer, numerous studies have been performed in attempts to further understand the mechanisms and effects of adriamycin. In one study, investigators injected mice with a single dose of adriamycin (Chen et al. (1998) Nephron 78:440-452). The mice exhibited signs of combined glomerular albuminuria and immunoglublinuria, progressively elevated levels of nitrite/nitrate in the urine, abnormal renal function, and other symptoms indicative of focal segmental glomerulosclerosis.

In another study, rats were given adriamycin and the effects on angiotensin converting enzyme (ACE) were monitored (Venkatesan et al. (1993) Toxicology 85:137-148). The rats developed glomerular and tubular injury, and serum ACE levels were significantly elevated 20, 25, and 30 days post-treatment. A different study followed rabbits for up to one year that were treated with either adriamycin, nephrectomy, or combinations thereof (Gadeholt-Gothlin et al. (1995) Urol Res 23:169-173). The rabbits that were treated with adriamycin exhibited signs of nephrotoxicity at relatively low doses.

Menadione (vitamin K₃) is a fat-soluble vitamin precursor that is converted into menaquinone in the liver. The primary known function of vitamin K is to assist in normal blood clotting, but it may also play a role in bone calcificaton. Menadione is a quinone compound that induces oxidative stress. It has been used as an anticancer agent and radiosensitizer and can produce toxicity in the kidney, lung, heart, and liver. In the kidney, signs of toxicity are dose-dependent, ranging from minor degranulation of tubular cells at lower doses to tubular dilatation, formation of protein casts in the renal tubules, calcium mineralization, vacuolization in the proximal and distal renal tubules, granular degeneration in the cortex and necrosis and apoptosis (Chiou et al., Toxicology 1997) 124(3):193-202).

Monocrotaline, an alkaloid obtained from Crotalaria spectabilis, a warm-climate garden plant, induces multi-organ toxicity affecting the kidney, heart, liver and lung. This compound is used to induce mesangiolysis in the kidney, to mimic the effects of Habu venom poisoning and hemolytic-uremic syndrome. Renal lesions in rats first appeared in the glomerular capillaries (endothelial cell detachment and adhesion of platelets to the basal lamina), followed by severe edema in the mesangium. Mesangiolysis subsequently occurred, accompanied by dilatation or obliteration of capillaries and necrosis and hemorrhage in the mesangium (Kurozumi et al., Exp Mol Pathol (1983) 39(3):377-386).

Vancomycin is a polycyclic glycoprotein antibiotic that is used to treat severe systemic infections by beta-lactam-resistant bacteria, in particular, resistant staphylococci. This drug may be given to patients who are allergic to penicillin. Vancomycin can induce renal failure and interstitial nephritis (Physicians Desk Reference 56th Ed., pp. 1970-1971, Medical Economics Co., Montvale, N.J., 2002).

Sodium chromate, a model compound used to induce liver toxicity, also produces toxic effects in the kidney. Necrosis of the S1 segment of the proximal tubule has been reported, as well as acute renal failure, characterized by increased levels of kininogens in the renal cortex and medulla and in urine and decreased rates of glomerular filtration (Bompart et al., Nephron (1993) 65(4):612-618; Beckwith-Hall et al, Chem Res Toxicol (1998) 11(4):260-272).

In the kidney, sodium oxalate forms crystals in the urinary tract, resulting in tubular obstruction, and produces calcific kidney stones in humans and in rats. The stones are located on renal papillary surfaces and consist of an organic matrix and crystals of calcium oxalate and/or calcium phosphate. The matrix is intimately associated with the crystals and contains substances that both promote and inhibit calcification: osteopontin, Tamm-Horsfall protein, bikunin and prothrombin fragment 1. Rats with these stones show decreased urine levels of magnesium and citrate, and the same is believed to occur in humans. Males of both species tend to develop calcium oxalate kidney stones, whereas females tend to form calcium phosphate stones (Khan, World J Urol (1997) 15(4):236-243).

Hexachloro-1,2-butadiene (HCBD) is a solvent that forms toxic conjugates and metabolites with glutathione, cysteine and N-acetyl cysteine. These then cause damage to the S1, S2 and S3 (pars recta) segments of the proximal tubules in the outer medulla of the kidney. Mitochondrial swelling has been observed in the S1 and S2 segments, although most of the pathological changes occur in the S2 and S3 segments (loss of brush boarder and cellular necrosis in S2, necrosis in S3). In rats, HCBD is about four times more toxic to females than to males (Ishmael et al., Toxicol Pathol (1986) 14(2):258-262; Ishmael et al., J Pathol (1982) 138(2):99-113).

Chloroform (CHCl₃) is widely used in the manufacture of drugs, cosmetics, plastics and cleaning agents and is a contaminant by-product in chlorinated drinking water. Chloroform was also an early anesthetic used in humans, and, therefore, much is known regarding its toxicity. Exposure can induce liver and kidney damage and cardiac arrthymias.

Toxic levels of exposure in rodents are carcinogenic due to the chronic cycle of cell injury and repair that is induced, rather than because of direct genotoxic action. The injury to the liver and kidney are thought to occur by two different mechanisms related to its metabolism in the target organ. Studies have shown that the extent of liver and kidney damage and necrosis relates multiple factors including sex, strain, route of exposure and the vehicle used. In the kidney, biotransformation of chloroform by cytochrome P450 produces reactive intermediates, which damage mainly the renal proximal tubules. Typical signs of nephrotoxicity include proteinuria, glucosuria and increased BUN levels (Casarett & Doull's Toxicology: The Basic Science of Poisons 6th Ed., Klaasen, ed., Chap. 14, pp. 503-508, McGraw-Hill, New York, 2001; Smith et al., Toxicol Appl Pharmacol 70:467-479, 1983).

Diclofenac, a non-steroidal anti-inflammatory drug, is commonly administered to patients suffering from rheumatoid arthritis, osteoarthritis, and ankylosing spondylitis. Following oral administration, diclofenac is rapidly absorbed and then metabolized in the liver by cytochrome P450 isozyme of the CYC2C subfamily (Goodman & Gilman's The Pharmacological Basis of Therapeutics 9^{th} Ed., Hardman et al., eds., p. 637, McGraw Hill, New York, 1996). In addition, diclofenac is used topically to treat pain due to corneal damage (Jayamanne et al., Eye 11(Pt. 1):79-83, 1997; Dornic et al., Am J. Ophthalmol 125(5):719-721, 1998).

Metabolism of diclofenac in kidney tissue produces reactive oxygen species that can cause severe oxidative stress and genomic DNA fragmentation. Examination of diverse types of kidney cells for nuclei with apoptotic characteristics showed that such nuclei are found in the linings of the renal proximal and distal tubules. Additional toxic effects include elevated levels of BUN, malondialdehyde (MDA), SOD, and activated Ca²⁺—Mg²⁺-endonuclease (Hickey et al., Free Radic Biol Med (2001) 31(2):139-152).

Thioacetamide's only significant commercial use is as a replacement for hydrogen sulfide in qualitative analyses (IARC, Vol. 7, 1974). It has also been used as an organic solvent in the leather, textile and paper industries, as an accelerator in the vulcanization of buna rubber, and as a stabilizer of motor fuel. The primary routes of human exposure are inhalation and skin contact with products in which thioacetamide was used as a solvent (9th Report on Carcinogens, U.S. Dept. of Health and Human Services, Public Health Service, National Toxicology Program, http://ehp.niehs.nih.gov/roc/toc9.html).

In exposed rats, thioacetamide was shown to accumulate in the liver and kidney, resulting in elevated levels of serum total bilirubin, aspartate aminotransferase, alanine aminotransferase, BUN, creatinine and TNFα. Impaired clearance of the toxin and increased secretion of TNFα are related to the progression of toxic effects in the liver and kidney (Nakatani et al., Liver (2001) 21(1):64-70). Additional histological changes in kidney tissue include glomerular tuft collapse and interstitial haemorrhage (Caballero et al., Gut (2001) 48(1):34-40).

Toxicity Prediction and Modeling

The genes and gene expression information, gene expression profiles, as well as the portfolios and subsets of the genes provided in Tables 1-5, may be used to predict at least one toxic effect, including the nephrotoxicity of a test or unknown compound. As used, herein, at least one toxic effect includes, but is not limited to, a detrimental change in the physiological status of a cell or organism. The response may be, but is not required to be, associated with a particular pathology, such as tissue necrosis. Accordingly, the toxic effect includes effects at the molecular and cellular level. Nephrotoxicity is an effect as used herein and includes but is not limited to the pathologies of nephritis, tubular toxicity, kidney necrosis, glomerular and tubular injury, and focal segmental glomerulosclerosis. As used herein, a gene expression profile comprises any quantitative representation of the expression of at least one mRNA species in a cell sample or population and includes profiles made by various methods such as differential display, PCR, microarray and other hybridization analysis, etc.

In general, assays to predict the toxicity or nephrotoxicity of a test agent (or compound or multi-component composition) comprise the steps of exposing a cell population to the test compound, assaying or measuring the level of relative or absolute gene expression of one or more of the genes in Tables 1-5 and comparing the identified expression level(s) to the expression levels disclosed in the Tables and database(s) disclosed herein. Assays may include the measurement of the expression levels of about 2, 3, 4, 5, 6, 7, 8, 9, 10, 15, 20, 25, 30, 50, 75, 100 or more genes from Tables 1-5.

In the methods of the invention, the gene expression level for a gene or genes induced by the test agent, compound or compositions may be comparable to the levels found in the Tables or databases disclosed herein if the expression level varies within a factor of about 2, about 1.5 or about 1.0 fold. In some cases, the expression levels are comparable if the agent induces a change in the expression of a gene in the same direction (e.g., up or down) as a reference toxin.

The cell population that is exposed to the test agent, compound or composition may be exposed in vitro or in vivo. For instance, cultured or freshly isolated renal cells, in particular rat renal cells, may be exposed to the agent under standard laboratory and cell culture conditions. In another assay format, in vivo exposure may be accomplished by administration of the agent to a living animal, for instance a laboratory rat.

Procedures for designing and conducting toxicity tests in in vitro and in vivo systems are well known, and are described in many texts on the subject, such as Loomis et al., Loomis's Esstentials of Toxicology, 4th Ed., Academic Press, New York, 1996; Echobichon, The Basics of Toxicity Testing, CRC Press, Boca Raton, 1992; Frazier, editor, In Vitro Toxicity Testing, Marcel Dekker, New York, 1992; and the like.

In in vitro toxicity testing, two groups of test organisms are usually employed: One group serves as a control and the other group receives the test compound in a single dose (for acute toxicity tests) or a regimen of doses (for prolonged or chronic toxicity tests). Because, in some cases, the extraction of tissue as called for in the methods of the invention requires sacrificing the test animal, both the control group and the group receiving compound must be large enough to permit removal of animals for sampling tissues, if it is desired to observe the dynamics of gene expression through the duration of an experiment.

In setting up a toxicity study, extensive guidance is provided in the literature for selecting the appropriate test organism for the compound being tested, route of administration. dose ranges, and the like. Water or physiological saline (0.9% NaCl in water) is the solute of choice for the test compound since these solvents permit administration by a variety of routes. When this is not possible because of solubility limitations, vegetable oils such as corn oil or organic solvents such as propylene glycol may be used.

Regardless of the route of administration, the volume required to administer a given dose is limited by the size of the animal that is used. It is desirable to keep the volume of each dose uniform within and between groups of animals. When rats or mice are used, the volume administered by the oral route generally should not exceed about 0.005 ml per gram of animal. Even when aqueous or physiological saline solutions are used for parenteral injection the volumes that are tolerated are limited, although such solutions are ordinarily thought of as being innocuous. The intravenous LD₅₀ of distilled water in the mouse is approximately 0.044 ml per gram and that of isotonic saline is 0.068 ml per gram of mouse. In some instances, the route of administration to the test animal should be the same as, or as similar as possible to, the route of administration of the compound to man for therapeutic purposes.

When a compound is to be administered by inhalation, special techniques for, generating test atmospheres are necessary. The methods usually involve aerosolization or nebulization of fluids containing the compound. If the agent to be tested is a fluid that has an appreciable vapor pressure, it may be administered by passing air through the solution under controlled temperature conditions. Under these conditions, dose is estimated from the volume of air inhaled per unit time, the temperature of the solution, and the vapor pressure of the agent involved. Gases are metered from reservoirs. When particles of a solution are to be administered, unless the particle size is less than about 2 μm the particles will not reach the terminal alveolar sacs in the lungs. A variety of apparatuses and chambers are available to perform studies for detecting effects of irritant or other toxic endpoints when they are administered by inhalation. The preferred method of administering an agent to animals is via the oral route, either by intubation or by incorporating the agent in the feed.

When the agent is exposed to cells in vitro or in cell culture, the cell population to be exposed to the agent may be divided into two or more subpopulations, for instance, by dividing the population into two or more identical aliquots. In some preferred embodiments of the methods of the invention, the cells to be exposed to the agent are derived from kidney tissue. For instance, cultured or freshly isolated rat renal cells may be used.

The methods of the invention may be used generally to predict at least one toxic response, and, as described in the Examples, may be used to predict the likelihood that a compound or test agent will induce various specific kidney pathologies, such as nephritis, kidney necrosis, glomerular and tubular injury, focal segmental glomerulosclerosis, or other pathologies associated with at least one of the toxins herein described. The methods of the invention may also be used to determine the similarity of a toxic response to one or more individual compounds. In addition, the methods of the invention may be used to predict or elucidate the potential cellular pathways influenced, induced or modulated by the compound or test agent due to the similarity of the expression profile compared to the profile induced by a known toxin (see Tables 5-5L).

Diagnostic Uses for the Toxicity Markers

As described above, the genes and gene expression information or portfolios of the genes with their expression information as provided in Tables 1-5 may be used as diagnostic markers for the prediction or identification of the physiological state of tissue or cell sample that has been exposed to a compound or to identify or predict the toxic effects of a compound or agent. For instance, a tissue sample such as a sample of peripheral blood cells or some other easily obtainable tissue sample may be assayed by any of the methods described above, and the expression levels from a gene or genes from Tables 1-5 may be compared to the expression levels found in tissues or cells exposed to the toxins described herein. These methods may result in the diagnosis of a physiological state in the cell or may be used to identify the potential toxicity of a compound, for instance a new or unknown compound or agent. The comparison of expression data, as well as available sequence or other information may be done by researcher or diagnostician or may be done with the aid of a computer and databases as described below.

In another format, the levels of a gene(s) of Tables 1-5, its encoded protein(s), or any metabolite produced by the encoded protein may be monitored or detected in a sample, such as a bodily tissue or fluid sample to identify or diagnose a physiological state of an organism. Such samples may include any tissue or fluid sample, including urine, blood and easily obtainable cells such as peripheral lymphocytes.

Use of the Markers for Monitoring Toxicity Progression

As described above, the genes and gene expression information provided in Tables 1-5 may also be used as markers for the monitoring of toxicity progression, such as that found after initial exposure to a drug, drug candidate, toxin, pollutant, etc. For instance, a tissue or cell sample may be assayed by any of the methods described above, and the expression levels from a gene or genes from Tables 1-5 may be compared to the expression levels found in tissue or cells exposed to the renal toxins described herein. The comparison of the expression data, as well as available sequence or other information may be done by a researcher or diagnostician or may be done with the aid of a computer and databases.

Use of the Toxicity Markers for Drug Screening

According to the present invention, the genes identified in Tables 1-5 may be used as markers or drug targets to evaluate the effects of a candidate drug, chemical compound or other agent on a cell or tissue sample. The genes may also be used as drug targets to screen for agents that modulate their expression and/or activity. In various formats, a candidate drug or agent can be screened for the ability to stimulate the transcription or expression of a given marker or markers or to down-regulate or counteract the transcription or expression of a marker or markers. According to the present invention, one can also compare the specificity of a drug's effects by looking at the number of markers which the drug induces and comparing them. More specific drugs will have less transcriptional targets. Similar sets of markers identified for two drugs may indicate a similarity of effects.

Assays to monitor the expression of a marker or markers as defined in Tables 1-5 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, gene chips containing probes to one, two or more genes from Tables 1-5 may be used to directly monitor or detect changes in gene expression in the treated or exposed cell. Cell lines, tissues or other samples are first exposed to a test agent and in some instances, a known toxin, and the detected expression levels of one or more, or preferably 2 or more of the genes of Tables 1-5 are compared to the expression levels of those same genes exposed to a known toxin alone. Compounds that modulate the expression patterns of the known toxin(s) would be expected to modulate potential toxic physiological effects in vivo. The genes in Tables 1-5 are particularly appropriate markers in these assays as they are differentially expressed in cells upon exposure to a known renal toxin. Tables 1 and 2 disclose those genes that are differentially expressed upon exposure to the named toxins and their corresponding GenBank Accession numbers. Table 3 discloses the human homologues and the corresponding GenBank Accession numbers of the differentially expressed genes of Tables 1 and 2.

In another format, cell lines that contain reporter gene fusions between the open reading frame and/or the transcriptional regulatory regions of a gene in Tables 1-5 and any assayable fusion partner may be prepared. Numerous assayable fusion partners are known and readily available including the firefly luciferase gene and the gene encoding chloramphenicol acetyltransferase (Alam et al. (1990), Anal Biochem 188:245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

Additional assay formats may be used to monitor the ability of the agent to modulate the expression of a gene identified in Tables 1-5. For instance, as described above, mRNA expression may be monitored directly by hybridization of probes to the nucleic acids of the invention. Cell lines are exposed to the agent to be tested under appropriate conditions and time, and total RNA or mRNA is isolated by standard procedures such those disclosed in Sambrook et al. (Molecular Cloning: A Laboratory Manual, 2nd Ed., Cold Spring Harbor Laboratory Press, Cold Spring Harbor, N.Y., 1989).

In another assay format, cells or cell lines are first identified which express the gene products of the invention physiologically. Cells and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines may be transduced or transfected with an expression vehicle (e.g., a plasmid or viral vector) construct comprising an operable non-translated 5′-promoter containing end of the structural gene encoding the gene products of Tables 1-5 fused to one or more antigenic fragments or other detectable markers, which are peculiar to the instant gene products, wherein said fragments are under the transcriptional control of said promoter and are expressed as polypeptides whose molecular weight can be distinguished from the naturally occurring polypeptides or may further comprise an immunologically distinct or other detectable tag. Such a process is well known in the art (see Sambrook et al., supra).

Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells are disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the agent-contacted sample is then compared with the control samples (no exposure and exposure to a known toxin) where only the excipient is contacted with the cells and an increase or decrease in the immunologically generated signal from the agent-contacted sample compared to the control is used to distinguish the effectiveness and/or toxic effects of the agent.

Another embodiment of the present invention provides methods for identifying agents that modulate at least one activity of a protein(s) encoded by the genes in Tables 1-5. Such methods or assays may utilize any means of monitoring or detecting the desired activity.

In one format, the relative amounts of a protein (Tables 1-5) between a cell population that has been exposed to the agent to be tested compared to an un-exposed control cell population and a cell population exposed to a known toxin may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Agents that are assayed in the above methods can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see G. A. Grant in: Molecular Biology and Biotechnology, Meyers, ed., pp. 659-664, VCH Publishers, New York, 1995). A skilled artisan can readily recognize that there is no limit as to the structural nature of the agents of the present invention.

Nucleic Acid Assay Formats

The genes identified as being differentially expressed upon exposure to a known renal toxin (Tables 1-5) may be used in a variety of nucleic acid detection assays to detect or quantify the expression level of a gene or multiple genes in a given sample. The genes described in Tables 1-5 may also be used in combination with one or more additional genes whose differential expression is associate with toxicity in a cell or tissue. In preferred embodiments, the genes in Tables 1-5 may be combined with one or more of the genes described in prior and related application Ser. Nos. 10/152,319, filed May 22, 2002; 60/292,335, filed May 22, 2001; 60/297,523, filed Jun. 13, 2001; 60/298,925, filed Jun. 19, 2001; 60/303,810, filed Jul. 10, 2001; 60/303,807, filed Jul. 10, 2001; 60/303,808, filed Jul. 10, 2001; 60/315,047, filed Aug. 28, 2001; 60/324,928, filed Sep. 27, 2001; 60/330,867, filed Nov. 1, 2001; 60/330,462, filed Oct. 22, 2001; 60/331,805, filed Nov. 21, 2001; 60/336,144, filed Dec. 6, 2001; 60/340,873, filed Dec. 19, 2001; 60/357,843, filed Feb. 21, 2002; 60/357,842, filed Feb. 21, 2002; 60/357,844, filed Feb. 21, 2002; 60/364,134; 60/370,206 filed Mar. 15, 2002, filed Apr. 8, 2002; 60/370,247, filed Apr. 8, 2002; 60/370,144, filed Apr. 8, 2002; 60/371,679, filed Apr. 12, 2002; and 60/372,794, filed Apr. 17, 2002, all of which are incorporated by reference on page 1 of this application.

Any assay format to detect gene expression may be used. For example, traditional Northern blotting, dot or slot blot, nuclease protection, primer directed amplification, RT-PCR, semi- or quantitative PCR, branched-chain DNA and differential display methods may be used for detecting gene expression levels. Those methods are useful for some embodiments of the invention. In cases where smaller numbers of genes are detected, amplification based assays may be most efficient. Methods and assays of the invention, however, may be most efficiently designed with hybridization-based methods for detecting the expression of a large number of genes.

Any hybridization assay format may be used, including solution-based and solid support-based assay formats. Solid supports containing oligonucleotide probes for differentially expressed genes of the invention can be filters, polyvinyl chloride dishes, particles, beads, microparticles or silicon or glass based chips, etc. Such chips, wafers and hybridization methods are widely available, for example, those disclosed by Beattie (WO 95/11755).

Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 or more of such features on a single solid support. The solid support, or the area within which the probes are attached may be on the order of about a square centimeter. Probes corresponding to the genes of Tables 1-5 or from the related applications described above may be attached to single or multiple solid support structures, e.g., the probes may be attached to a single chip or to multiple chips to comprise a chip set.

Oligonucleotide probe arrays for expression monitoring can be made and used according to any techniques known in the art (see for example, Lockhart et al (1996), Nat Biotechnol 14:1675-1680; McGall et al. (1996), Proc Nat Acad Sci USA 93: 13555-13460). Such probe arrays may contain at least two or more oligonucleotides that are complementary to or hybridize to two or more of the genes described in Tables 1-5. For instance, such arrays may contain oligonucleotides that are complementary to or hybridize to at least 2, 3, 4, 5, 6, 7, 8, 9, 10, 20, 30, 50, 70, 100 or more of the genes described herein. Preferred arrays contain all or nearly all of the genes listed in Tables 1-5, or individually, the gene sets of Tables 5-5L. In a preferred embodiment, arrays are constructed that contain oligonucleotides to detect all or nearly all of the genes in any one of or all of Tables 1-5 on a single solid support substrate, such as a chip.

The sequences of the expression marker genes of Tables 1-5 are in the public databases. Table 1 provides the GenBank Accession Number or NCBI RefSeq ID for each of the sequences (see www.ncbi.nlm.nih.gov/), as well as the title for the cluster of which gene is part. Table 2 lists the metabolic pathways in which each listed gene functions, while Table 3 provides the gene names and cluster titles for the human homologues of the genes described in Tables 1 and 2. The sequences of the genes in GenBank and/or RefSeq are expressly herein incorporated by reference in their entirety as of the filing date of this application, as are related sequences, for instance, sequences from the same gene of different lengths, variant sequences, polymorphic sequences, genomic sequences of the genes and related sequences from different species, including the human counterparts, where appropriate. These sequences may be used in the methods of the invention or may be used to produce the probes and arrays of the invention. In some embodiments, the genes in Tables 1-5 that correspond to the genes or fragments previously associated with a toxic response may be excluded from the Tables. Table 4 provides the key to the model codes used in Tables 3 and 5-5L, where each model represents a toxin treatment or a set of pathological effects (disease state) resulting from a toxin treatment. In Tables 5A-5L, the genes that are differentially expressed, i.e., up- or down-regulated, in response to a toxin treatment or in a particular disease state are listed. The expression levels of these genes in samples in which a toxic response was found and in samples in which a toxic response was not found are also indicated. The general table (Table 5) is a summation of the data in Tables 5A-5L.

As described above, in addition to the sequences of the GenBank Accession Numbers or NCBI RefSeq ID's disclosed in the Tables 1-5, sequences such as naturally occurring variants or polymorphic sequences may be used in the methods and compositions of the invention. For instance, expression levels of various allelic or homologous forms of a gene disclosed in Tables 1-5 may be assayed, including homologs from species other than rat. Any and all nucleotide variations that do not alter the functional activity of a gene listed in the Tables 1-5, including all naturally occurring allelic variants of the genes herein disclosed, may be used in the methods and to make the compositions (e.g., arrays) of the invention.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes described in Tables 1-5 refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” or “specifically hybridizes” refers to the binding, duplexing, or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 1,000,000 different nucleic acid hybridizations.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.”

The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases.

While the mismatch(es) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical submit (e.g. nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Probe Design

One of skill in the art will appreciate that an enormous number of array designs are suitable for the practice of this invention. The high density array will typically include a number of test probes that specifically hybridize to the sequences of interest. Probes may be produced from any region of the genes identified in the Tables and the attached representative sequence listing. In instances where the gene reference in the Tables is an EST, probes may be designed from that sequence or from other regions of the corresponding full-length transcript that may be available in any of the sequence databases, such as those herein described. See WO 99/32660 for methods of producing probes for a given gene or genes. In addition, any available software may be used to produce specific probe sequences, including, for instance, software available from Molecular Biology Insights, Olympus Optical Co. and Biosoft International. In a preferred embodiment, the array will also include one or more control probes.

High density array chips of the invention include “test probes.” Test probes may be oligonucleotides that range from about 5 to about 500, or about 7 to about 50 nucleotides, more preferably from about 10 to about 40 nucleotides and most preferably from about 15 to about 35 nucleotides in length. In other particularly preferred embodiments, the probes are 20 or 25 nucleotides in length. In another preferred embodiment, test probes are double or single strand DNA sequences such as cDNA fragments. DNA sequences are isolated or cloned from natural sources or amplified from natural sources using native nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes may fall into three categories referred to herein as 1) normalization controls; 2) expression level controls; and 3) mismatch controls.

Normalization controls are oligonucleotide or other nucleic acid probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present the perfect match probes should be consistently brighter than the mismatch probes. In addition, if all central mismatches are present, the mismatch probes can be used to detect a mutation, for instance, a mutation of a gene in the accompanying Tables 1-5. The difference in intensity between the perfect match and the mismatch probe provides a good measure of the concentration of the hybridized material.

Nucleic Acid Samples

Cell or tissue samples may be exposed to the test agent in vitro or in vivo. When cultured cells or tissues are used, appropriate mammalian cell extracts, such as liver extracts, may also be added with the test agent to evaluate agents that may require biotransformation to exhibit toxicity. In a preferred format, primary isolates or cultured cell lines of animal or human renal cells may be used.

The genes which are assayed according to the present invention are typically in the form of mRNA or reverse transcribed mRNA. The genes may or may not be cloned. The genes may or may not be amplified. The cloning and/or amplification do not appear to bias the representation of genes within a population. In some assays, it may be preferable, however, to use polyA+ RNA as a source, as it can be used with less processing steps.

As is apparent to one of ordinary skill in the art, nucleic acid samples used in the methods and assays of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology Vol. 24, Hybridization With Nucleic Acid Probes: Theory and Nucleic Acid Probes, P. Tijssen, Ed., Elsevier Press, New York, 1993. Such samples include RNA samples, but also include cDNA synthesized from a mRNA sample isolated from a cell or tissue of interest. Such samples also include DNA amplified from the cDNA, and RNA transcribed from the amplified DNA. One of skill in the art would appreciate that it is desirable to inhibit or destroy RNase present in homogenates before homogenates are used.

Biological samples may be of any biological tissue or fluid or cells from any organism as well as cells raised in vitro, such as cell lines and tissue culture cells. Frequently the sample will be a tissue or cell sample that has been exposed to a compound, agent, drug, pharmaceutical composition, potential environmental pollutant or other composition. In some formats, the sample will be a “clinical sample” which is a sample derived from a patient. Typical clinical samples include, but are not limited to, sputum, blood, blood-cells (e.g., white cells), tissue or fine needle biopsy samples, urine, peritoneal fluid, and pleural fluid, or cells therefrom. Biological samples may also include sections of tissues, such as frozen sections or formalin fixed sections taken for histological purposes.

Forming High Density Arrays

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithographic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing. See WO 99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency, in this case in 6×SSPET at 37° C. (0.005% Triton X-100), to ensure hybridization and then subsequent washes are performed at higher stringency (e.g., 1×SSPET at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art. See WO 99/32660.

Databases

The present invention includes relational databases containing sequence information, for instance, for the genes of Tables 1-5, as well as gene expression information from tissue or cells exposed to various standard toxins, such as those herein described (see Tables 5-5L). Databases may also contain information associated with a given sequence or tissue sample such as descriptive information about the gene associated with the sequence information (see Tables 1 and 2), or descriptive information concerning the clinical status of the tissue sample, or the animal from which the sample was derived. The database may be designed to include different parts, for instance a sequence database and a gene expression database. Methods for the configuration and construction of such databases and computer-readable media to which such databases are saved are widely available, for instance, see U.S. Pat. No. 5,953,727, which is herein incorporated by reference in its entirety.

The databases of the invention may be linked to an outside or external database such as GenBank (www.ncbi.nlm.nih.gov/entrez.index.html); KEGG (www.genome.ad.jplkegg); SPAD (www.grt.kyushu-u.acjplspadlindex.html); HUGO (www.gene.ucl.ac.uk/hugo); Swiss-Prot (www.expasy.ch.sprot); Prosite (www.expasy.ch/tools/scnpsitl.html); OMIM (www.nebi.nlm.nih.gov/omim); and GDB (www.gdb.org). In a preferred embodiment, as described in Tables 1-5, the external database is GenBank and the associated databases maintained by the National Center for Biotechnology Information (NCBI) (www.ncbi.nlm.nih.gov).

Any appropriate computer platform, user interface, etc. may be used to perform the necessary comparisons between sequence information, gene expression information and any other information in the database or information provided as an input. For example, a large number of computer workstations are available from a variety of manufacturers, such has those available from Silicon Graphics. Client/server environments, database servers and networks are also widely available and appropriate platforms for the databases of the invention.

The databases of the invention may be used to produce, among other things, electronic Northerns that allow the user to determine the cell type or tissue in which a given gene is expressed and to allow determination of the abundance or expression level of a given gene in a particular tissue or cell.

The databases of the invention may also be used to present information identifying the expression level in a tissue or cell of a set of genes comprising one or more of the genes in Tables 1-5, comprising the step of comparing the expression level of at least one gene in Tables 1-5 in a cell or tissue exposed to a test agent to the level of expression of the gene in the database. Such methods may be used to predict the toxic potential of a given compound by comparing the level of expression of a gene or genes in Tables 1-5 from a tissue or cell sample exposed to the test agent to the expression levels found in a control tissue or cell samples exposed to a standard toxin or renal toxin such as those herein described. Such methods may also be used in the drug or agent screening assays as described herein.

Kits

The invention further includes kits combining, in different combinations, high-density oligonucleotide arrays, reagents for use with the arrays, protein reagents encoded by the genes of the Tables, signal detection and array-processing instruments, gene expression databases and analysis and database management software described above. The kits may be used, for example, to predict or model the toxic response of a test compound, to monitor the progression of renal disease states, to identify genes that show promise as new drug targets and to screen known and newly designed drugs as discussed above.

The databases packaged with the kits are a compilation of expression patterns from human or laboratory animal genes and gene fragments (corresponding to the genes of Tables 1-5). In particular, the database software and packaged information that may contain the databases saved to a computer-readable medium include the expression results of Tables 1-5 that can be used to predict toxicity of a test agent by comparing the expression levels of the genes of Tables 1-5 induced by the test agent to the expression levels presented in Tables 5-5L. In another format, database and software information may be provided in a remote electronic format, such as a website, the address of which may be packaged in the kit.

The kits may used in the pharmaceutical industry, where the need for early drug testing is strong due to the high costs associated with drug development, but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Databases and software designed for use with microarrays is discussed in Balaban et al., U.S. Pat. No. 6,229,911, a computer-implemented method for managing information, stored as indexed Tables 1-5, collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164, disclose a software-based method for identifying mutations in a nucleic acid sequence based on differences in probe fluorescence intensities between wild type and mutant sequences that hybridize to reference sequences.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Identification of Toxicity Markers

The renal toxins indomethacin, diflunisal, colchicine, chloroform, diclofenac, menadione, sodium chromate, sodium oxalate, thioacetamide, and vancomycin were administered to male Sprague-Dawley rats at various timepoints using administration diluents, protocols and dosing regimes as previously described in the art and previously described in the priority applications discussed above. In experiments using toxins A-G, as labeled in Table 4, blood and tissue samples were collected at the following time-points: chloroform (A), thioacetamide (F) and vancomycin (G)—after 6, 24 and 48 hours of exposure; diclofenac (B) and menadione (C)—after 3, 6 and 24 hours of exposure; and sodium chromate (D) and sodium oxalate (E)—after 6, 24 and 72 hours of exposure. For these compounds, no significant changes in the levels of gene expression were found with varying exposure time, i.e., short and long time-points showed the same pattern of differential gene expression. The low and high dose level for each compound are provided in the chart below.

| | Low Dose | | Method of |
|---|---|---|---|
| Renal Toxin | (mg/kg) | High Dose (mg/kg) | Administration |
| indomethacin | 1 | 10 | oral gavage |
| diflunisal | 2 | 400 | oral gavage |
| colchicine | 0.15 | 1.5 | intraperitoneal |
| chloroform | 11.95 | 239 | oral gavage |
| diclofenac | 1 | 200 | intraperitoneal |
| menadione | 15 | 150 | intravenous |
| sodium chromate | 3 | 30 | intraperitoneal |
| sodium oxalate | 10 | 100 | intraperitoneal |
| thioacetamide | 30 | 300 | intraperitoneal |
| vancomycin | 50 | 500 | intravenous |

After administration, the dosed animals were observed and tissues were collected as described below:

Observation of Animals

1. Clinical cage side observations—twice daily mortality and moribundity check. Skin and fur, eyes and mucous membrane, respiratory system, circulatory system, autonomic and central nervous system, somatomotor pattern, and behavior pattern were checked. Potential signs of toxicity, including tremors, convulsions, salivation, diarrhea, lethargy, coma or other atypical behavior or appearance, were recorded as they occurred and included a time of onset, degree, and duration.

2. Physical Examinations—Prior to randomization, prior to initial treatment, and prior to sacrifice.

3. Body Weights—Prior to randomization, prior to initial treatment, and prior to sacrifice.

Clinical Pathology

1. Frequency—Prior to necropsy.

2. Number of animals—All surviving animals.

3. Bleeding Procedure—Blood was obtained by puncture of the orbital sinus while under 70% CO₂/30% O₂ anesthesia.

4. Collection of Blood Samples—Approximately 0.5 mL of blood was collected into EDTA tubes for evaluation of hematology parameters. Approximately 1 mL of blood was collected into serum separator tubes for clinical chemistry analysis. Approximately 200 uL of plasma was obtained and frozen at ˜−80° C. for test compound/metabolite estimation. An additional ˜2 mL of blood was collected into a 15 mL conical polypropylene vial to which ˜3 mL of Trizol was immediately added. The contents were immediately mixed with a vortex and by repeated inversion. The tubes were frozen in liquid nitrogen and stored at ˜−80° C.

Termination Procedures

Terminal Sacrifice

Approximately 3, 6, 24, 48, 72, 120, 144, 168, 336, and/or 360 hours after the initial dose, rats were weighed, physically examined, sacrificed by decapitation, and exsanguinated. The animals were necropsied within approximately five minutes of sacrifice. Separate sterile, disposable instruments were used for each animal, with the exception of bone cutters, which were used to open the skull cap. The bone cutters were dipped in disinfectant solution between animals.

Necropsies were conducted on each animal following procedures approved by board-certified pathologists.

Animals not surviving until terminal sacrifice were discarded without necropsy (following euthanasia by carbon dioxide asphyxiation, if moribund). The approximate time of death for moribund or found dead animals was recorded.

Postmortem Procedures

Fresh and sterile disposable instruments were used to collect tissues. Gloves were worn at all times when handling tissues or vials. All tissues were collected and frozen within approximately 5 minutes of the animal's death. The liver sections and kidneys were frozen within approximately 3-5 minutes of the animal's death. The time of euthanasia, an interim time point at freezing of liver sections and kidneys, and time at completion of necropsy were recorded. Tissues were stored at approximately −80° C. or preserved in 10% neutral buffered formalin.

Tissue Collection and Processing

Liver

1. Right medial lobe—snap frozen in liquid nitrogen and stored at ˜−80° C.

2. Left medial lobe—Preserved in 10% neutral-buffered formalin (NBF) and evaluated for gross and microscopic pathology.

3. Left lateral lobe—snap frozen in liquid nitrogen and stored at ˜−80° C.

Heart—A sagittal cross-section containing portions of the two atria and of the two ventricles was preserved in 10% NBF. The remaining heart was frozen in liquid nitrogen and stored at ˜−80° C.

Kidneys (Both)

1. Left—Hemi-dissected; half was preserved in 10% NBF and the remaining half was frozen in liquid nitrogen and stored at ˜−80° C.

2. Right—Hemi-dissected; half was preserved in 10% NBF and the remaining half was frozen in liquid nitrogen and stored at ˜−80° C.

Testes (both)—A sagittal cross-section of each testis was preserved in 10% NBF. The remaining testes were frozen together in liquid nitrogen and stored at ˜−80° C.

Brain (whole)—A cross-section of the cerebral hemispheres and of the diencephalon was preserved in 10% NBF, and the rest of the brain was frozen in liquid nitrogen and stored at ˜−80° C.

Microarray sample preparation was conducted with minor modifications, following the protocols set forth in the Affymetrix GeneChip Expression Analysis Manual. Frozen tissue was ground to a powder using a Spex Certiprep 6800 Freezer Mill. Total RNA was extracted with Trizol (GibcoBRL) utilizing the manufacturer's protocol. The total RNA yield for each sample was 200-500 μg per 300 mg tissue weight. mRNA was isolated using the Oligotex mRNA Midi kit (Qiagen) followed by ethanol precipitation. Double stranded cDNA was generated from mRNA using the SuperScript Choice system (GibcoBRL). First strand cDNA synthesis was primed with a T7-(dT24) oligonucleotide. The cDNA was phenol-chloroform extracted and ethanol precipitated to a final concentration of 1 μg/ml. From 2 μg of cDNA, cRNA was synthesized using Ambion's T7 MegaScript in vitro Transcription Kit.

To biotin label the cRNA, nucleotides Bio-11-CTP and Bio-16-UTP (Enzo Diagnostics) were added to the reaction. Following a 37° C. incubation for six hours, impurities were removed from the labeled cRNA following the RNeasy Mini kit protocol (Qiagen). cRNA was fragmented (fragmentation buffer consisting of 200 mM Tris-acetate, pH 8.1, 500 mM KOAc, 150 mM MgOAc) for thirty-five minutes at 94° C. Following the Affymetrix protocol, 55 μg of fragmented cRNA was hybridized on the Affymetrix rat array set for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Data was analyzed using Affymetrix GeneChip® version 2.0 and Expression Data Mining (EDMT) software (version 1.0), GeneExpress2000, and S-Plus.

Tables 1 and 2 disclose those genes that are differentially expressed upon exposure to the named toxins and their corresponding GenBank Accession and Sequence Identification numbers, the identities of the metabolic pathways in which the genes function, the gene names if known, and the unigene cluster titles. The model code represents the various toxicity state that each gene is able to discriminate as well as the individual toxin type associated with each gene. The codes are defined in Table 4. The GLGC ID is the internal Gene Logic identification number.

Table 3 discloses those genes that are the human homologues of those genes in Tables 1 and 2 that are differentially expressed upon exposure to the named toxins. The corresponding GenBank Accession and Sequence Identification numbers, the gene names if known, and the unigene cluster titles of the human homologues are listed.

Table 4 defines the comparison codes used in Tables 1, 2, 3, and 5.

Tables 5-5L disclose the summary statistics for each of the comparisons performed. Each of these tables contains a set of predictive genes and creates a model for predicting the renal toxicity of an unknown, i.e., untested compound. Each gene is identified by its Gene Logic identification number and can be cross-referenced to a gene name and representative SEQ ID NO. in Tables 1 and 2. For each comparison of gene expression levels between samples in the toxicity group (samples affected by exposure to a specific toxin) and samples in the non-toxicity group (samples not affected by exposure to that same specific toxin), the tox mean (for toxicity group samples) is the mean signal intensity, as normalized for the various chip parameters that are being assayed. The non-tox mean represents the mean signal intensity, as normalized for the various chip parameters that are being assayed, in samples from animals other than those treated with the high dose of the specific toxin. These animals were treated with a low dose of the specific toxin, or with vehicle alone, or with a different toxin. Samples in the toxicity groups were obtained from animals sacrificed at the timepoint(s) indicated in the Table 5 headings, while samples in the non-toxicity groups were obtained from animals sacrificed at all time points in the experiments. For individual genes, an increase in the tox mean compared to the non-tox mean indicates up-regulation upon exposure to a toxin. Conversely, a decrease in the tox mean compared to the non-tox mean indicates down-regulation.

The mean values are derived from Average Difference (AveDiff) values for a particular gene, averaged across the corresponding samples. Each individual Average Difference value is calculated by integrating the intensity information from multiple probe pairs that are tiled for a particular fragment. The normalization multiplies each expression intensity for a given experiment (chip) by a global scaling factor. The intent of this normalization is to make comparisons of individual genes between chips possible. The scaling factor is calculated as follows:

1. From all the unnormalized expression values in the experiment, delete the largest 2% and smallest 2% of the values. That is, if the experiment yields 10,000 expression values, order the values and delete the smallest 200 and the largest 200.

2. Compute the trimmed mean, which is equal to the mean of the remaining values.

3. Compute the scale factor SF=100/(trimmed mean).

The value of 100 used here is the standard target value used. Some AveDiff values may be negative due to the general noise involved in nucleic acid hybridization experiments. Although many conclusions can be made corresponding to a negative value on the GeneChip platform, it is difficult to assess the meaning behind the negative value for individual fragments. Our observations show that, although negative values are observed at times within the predictive gene set, these values reflect a real biological phenomenon that is highly reproducible across all the samples from which the measurement was taken. For this reason, those genes that exhibit a negative value are included in the predictive set. It should be noted that other platforms of gene expression measurement may be able to resolve the negative numbers for the corresponding genes. The predictive ability of each of those genes should extend across platforms, however. Each mean value is accompanied by the standard deviation for the mean. The linear discriminant analysis score (discriminant score), as disclosed in the tables, measures the ability of each gene to predict whether or not a sample is toxic. The discriminant score is calculated by the following steps:

Calculation of a Discriminant Score

1. Let Xᵢ represent the AveDiff values for a given gene across the non-tox samples, i=1 . . . n.

2. Let Yᵢ represent the AveDiff values for a given gene across the tox samples, i=1 . . . t. The calculations proceed as follows:

3. Calculate mean and standard deviation for Xᵢ's and Yᵢ's, and denote these by m_{X}, m_{Y}, s_{X},s_{Y}.

4. For all Xᵢ's and Y₁'s, evaluate the function f(z)=((1/s_{Y})*exp(−0.5*((z-m_{Y})/s_{Y})²))/(((1/s_{Y})*exp(−0.5*((z-m_{Y})/s_{Y})²))+((1/s_{X})*exp(−0.5*((Z-m_{X})/s_{X})²))).

5. The number of correct predictions, say P, is then the number of Y₁'s such that f(Y₁)>0.5 plus the number of X₁'s such that f(X₁)<0.5.

6. The discriminant score is then P/(n+t).

Linear discriminant analysis uses both the individual measurements of each gene and the calculated measurements of all combinations of genes to classify samples. For each gene a weight is derived from the mean and standard deviation of the toxic and nontox groups. Every gene is multiplied by a weight and the sum of these values results in a collective discriminate score. This discriminant score is then compared against collective centroids of the tox and nontox groups. These centroids are the average of all tox and nontox samples respectively. Therefore, each gene contributes to the overall prediction. This contribution is dependent on weights that are large positive or negative numbers if the relative distances between the tox and nontox samples for that gene are large and small numbers if the relative distances are small. The discriminant score for each unknown sample and centroid values can be used to calculate a probability between zero and one as to the group in which the unknown sample belongs.

### Example 2

### General Toxicity Modeling

Samples were selected for grouping into tox-responding and non-tox-responding groups by examining each study individually with Principal Components Analysis (PCA) to determine which treatments had an observable response. Only groups where confidence of their tox-responding and non-tox-responding status was established were included in building a general tox model (Table 5).

Linear discriminant models were generated to describe toxic and non-toxic samples. The top discriminant genes and/or EST's were used to determine toxicity by calculating each gene's contribution with homo and heteroscedastic treatment of variance and inclusion or exclusion of mutual information between genes. Prediction of samples within the database exceeded 80% true positives with a false positive rate of less than 5%. It was determined that combinations of genes and/or EST's generally provided a better predictive ability than individual genes and that the more genes and/or EST used the better predictive ability. Although the preferred embodiment includes fifty or more genes, many pairings or greater combinations of genes and/or EST can work better than individual genes. All combinations of two or more genes from the selected list (Table 5) could be used to predict toxicity. These combinations could be selected by pairing in an agglomerate, divisive, or random approach. Further, as yet undetermined genes and/or EST's could be combined with individual or combination of genes and/or EST's described here to increase predictive ability. However, the genes and/or EST's described here would contribute most of the predictive ability of any such undetermined combinations.

Other variations on the above method can provide adequate predictive ability. These include selective inclusion of components via agglomerate, divisive, or random approaches or extraction of loading and combining them in agglomerate, divisive, or random approaches. Also the use of composite variables in logistic regression to determine classification of samples can also be accomplished with linear discriminate analysis, neural or Bayesian networks, or other forms of regression and classification based on categorical or continual dependent and independent variables.

### Example 3

### Modeling Methods

The above modeling methods provide broad approaches of combining the expression of genes to predict sample toxicity. One could also provide no weight in a simple voting method or determine weights in a supervised or unsupervised method using agglomerate, divisive, or random approaches. All or selected combinations of genes may be combined in ordered, agglomerate, or divisive, supervised or unsupervised clustering algorithms with unknown samples for classification. Any form of correlation matrix may also be used to classify unknown samples. The spread of the group distribution and discriminate score alone provide enough information to enable a skilled person to generate all of the above types of models with accuracy that can exceed discriminate ability of individual genes. Some examples of methods that could be used individually or in combination after transformation of data types include but are not limited to: Discriminant Analysis, Multiple Discriminant Analysis, logistic regression, multiple regression analysis, linear regression analysis, conjoint analysis, canonical correlation, hierarchical cluster analysis, k-means cluster analysis, self-organizing maps, multidimensional scaling, structural equation modeling, support vector machine determined boundaries, factor analysis, neural networks, bayesian classifications, and resampling methods.

### Example 4

### Grouping of Individual Compound and Pathology Classes

Samples were grouped into individual pathology classes based on known toxicological responses and observed clinical chemical and pathology measurements or into early and late phases of observable toxicity within a compound (Tables 5A-5L). The top 10, 25, 50, 100 genes based on individual discriminate scores were used in a model to ensure that combination of genes provided a better prediction than individual genes. As described above, all combinations of two or more genes from this list could potentially provide better prediction than individual genes when selected in any order or by ordered, agglomerate, divisive, or random approaches. In addition, combining these genes with other genes could provide better predictive ability, but most of this predictive ability would come from the genes listed herein.

Samples may be considered toxic if they score positive in any pathological or individual compound class represented here or in any modeling method mentioned under general toxicology models based on combination of individual time and dose grouping of individual toxic compounds obtainable from the data. The pathological groupings and early and late phase models are preferred examples of all obtainable combinations of sample time and dose points. Most logical groupings with one or more genes and one or more sample dose and time points should produce better predictions of general toxicity, pathological specific toxicity, or similarity to known toxicant than individual genes.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

LENGTHY TABLE REFERENCED HERE
US20070015146A1-20070118-T00001
Please refer to the end of the specification for access instructions.

LENGTHY TABLE REFERENCED HERE
US20070015146A1-20070118-T00002
Please refer to the end of the specification for access instructions.

LENGTHY TABLE REFERENCED HERE
US20070015146A1-20070118-T00003
Please refer to the end of the specification for access instructions.

LENGTHY TABLE REFERENCED HERE
US20070015146A1-20070118-T00004
Please refer to the end of the specification for access instructions.

LENGTHY TABLE REFERENCED HERE
US20070015146A1-20070118-T00005
Please refer to the end of the specification for access instructions.

LENGTHY TABLE REFERENCED HERE
US20070015146A1-20070118-T00006
Please refer to the end of the specification for access instructions.

LENGTHY TABLE REFERENCED HERE
US20070015146A1-20070118-T00007
Please refer to the end of the specification for access instructions.

LENGTHY TABLE REFERENCED HERE
US20070015146A1-20070118-T00008
Please refer to the end of the specification for access instructions.

LENGTHY TABLE REFERENCED HERE
US20070015146A1-20070118-T00009
Please refer to the end of the specification for access instructions.

LENGTHY TABLE REFERENCED HERE
US20070015146A1-20070118-T00010
Please refer to the end of the specification for access instructions.

LENGTHY TABLE REFERENCED HERE
US20070015146A1-20070118-T00011
Please refer to the end of the specification for access instructions.

LENGTHY TABLE REFERENCED HERE
US20070015146A1-20070118-T00012
Please refer to the end of the specification for access instructions.

LENGTHY TABLE REFERENCED HERE
US20070015146A1-20070118-T00013
Please refer to the end of the specification for access instructions.

LENGTHY TABLE REFERENCED HERE
US20070015146A1-20070118-T00014
Please refer to the end of the specification for access instructions.

LENGTHY TABLE REFERENCED HERE
US20070015146A1-20070118-T00015
Please refer to the end of the specification for access instructions.

LENGTHY TABLE REFERENCED HERE
US20070015146A1-20070118-T00016
Please refer to the end of the specification for access instructions.

LENGTHY TABLE REFERENCED HERE
US20070015146A1-20070118-T00017
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070015146A1) An electronic copy of the table will also be available from the USPTO upon request and payment of the fee set forth in 37 CFR 1.19(b)(3).

We claim:

1. A method of predicting at least one toxic effect of a compound, comprising:
(a) preparing a gene expression profile of a tissue or cell sample exposed to the compound; and
(b) comparing the gene expression profile to a database comprising at least part of the data or information of Tables 1-5.

2. A method of claim 1, wherein the gene expression profile prepared from the tissue or cell sample comprises the level of expression for at least one gene.

3. A method of claim 2, wherein the level of expression is compared to a Tox Mean and/or NonTox Mean value in Tables 5-5L.

4. A method of claim 3, wherein the level of expression is normalized prior to comparison.

5. A method of claim 1, wherein the database comprises substantially all of the data or information in Tables 5-5L.

6. A method of predicting at least one toxic effect of a compound, comprising:
(a) detecting the level of expression in a tissue or cell sample exposed to the compound of two or more genes from Tables 1-5; wherein differential expression of the genes in Tables 1-5 is indicative of at least one toxic effect.

7. A method of predicting the progression of a toxic effect of a compound, comprising:
(a) detecting the level of expression in a tissue or cell sample exposed to the compound of two or more genes from Tables 1-5; wherein differential expression of the genes in Tables 1-5 is indicative of toxicity progression.

8. A method of predicting the renal toxicity of a compound, comprising:
(a) detecting the level of expression in a tissue or cell sample exposed to the compound of two or more genes from Tables 1-5; wherein differential expression of the genes in Tables 1-5 is indicative of renal toxicity.

9. A method of identifying an agent that modulates the onset or progression of a toxic response, comprising:
(a) exposing a cell to the agent and a known toxin; and
(b) detecting the expression level of two or more genes from Tables 1-5; wherein differential expression of the genes in Tables 1-5 is indicative of toxicity.

10. A method of predicting the cellular pathways that a compound modulates in a cell, comprising:
(a) detecting the level of expression in a tissue or cell sample exposed to the compound of two or more genes from Tables 1-5; wherein differential expression of the genes in Tables 1-5 is associated the modulation of at least one cellular pathway.

11. The method of claim 6, wherein the expression levels of at least 3 genes are detected.

12. The method of claim 6, wherein the expression levels of at least 4 genes are detected.

13. The method of claim 6, wherein the expression levels of at least 5 genes are detected.

14. The method of claim 6, wherein the expression levels of at least 6 genes are detected.

15. The method of claim 6, wherein the expression levels of at least 7 genes are detected.

16. The method of claim 6, wherein the expression levels of at least 8 genes are detected.

17. The method of claim 6, wherein the expression levels of at least 9 genes are detected.

18. The method of claim 6, wherein the two or more genes are rat genes.

19. A method of claim 6, wherein the effect is selected from the group consisting of nephritis, kidney necrosis, glomerular and tubular injury, and focal segmental glomerulosclerosis.

20. A method of claim 8, wherein the renal toxicity is associated with at least one kidney disease pathology selected from the group consisting of nephritis, kidney necrosis, glomerular and tubular injury, and focal segmental glomerulosclerosis.

21. A method of claim 10, wherein the cellular pathway is modulated by a toxin selected from the group consisting of indomethacin, diflunisal, colchicine, chloroform, diclofenac, menadione, sodium chromate, sodium oxalate, thioacetamide and vancomycin.

22. A set of at least two probes, wherein each of the probes comprises a sequence that specifically hybridizes to a gene in Tables 1-5.

23. A set of probes according to claim 22, wherein the set comprises probes that hybridize to at least 3 genes.

24. A set of probes according to claim 22, wherein the set comprises probes that hybridize to at least 5 genes.

25. A set of probes according to claim 22, wherein the set comprises probes that hybridize to at least 7 genes.

26. A set of probes according to claim 22, wherein the set comprises probes that hybridize to at least 10 genes.

27. A set of probes according to any one of claims 22-26, wherein the probes are attached to a solid support.

28. A set of probes according to claim 27, wherein the solid support is selected from the group consisting of a membrane, a glass support and a silicon support.

29. A solid support comprising at least two probes, wherein each of the probes comprises a sequence that specifically hybridizes to a gene in Tables 1-5.

30. A solid support of claim 29, wherein the gene is a rat gene.

31. A solid support of claim 29, wherein the array comprises at least about 100 different oligonucleotides in discrete locations per square centimeter.

32. A solid support of claim 29, wherein the array comprises at least about 1000 different oligonucleotides in discrete locations per square centimeter.

33. A solid support of claim 29, wherein the array comprises at least about 10,000 different oligonucleotides in discrete locations per square centimeter.

34. A computer system comprising:
(a) a database containing information identifying the expression level in a tissue or cell sample exposed to a renal toxin of a set of genes comprising at least two genes in Tables 1-5; and
(b) a user interface to view the information.

35. A computer system of claim 34, wherein the database further comprises sequence information for the genes.

36. A computer system of claim 34, wherein the database further comprises information identifying the expression level for the set of genes in the tissue or cell sample before exposure to a renal toxin.

37. A computer system of claim 34, wherein the database further comprises information identifying the expression level of the set of genes in a tissue or cell sample exposed to at least a second renal toxin.

38. A computer system of claim 34, further comprising records including descriptive information from an external database, which information correlates said genes to records in the external database.

39. A computer system of claim 38, wherein the external database is GenBank.

40. A method of using a computer system of any one of claims 34-37 to present information identifying the expression level in a tissue or cell of at least one gene in Tables 1-5, comprising:
comparing the expression level of at least one gene in Tables 1-5 in a tissue or cell exposed to a test agent to the level of expression of the gene in the database.

41. A method of claim 40, wherein the expression levels of at least two genes are compared.

42. A method of claim 40, wherein the expression levels of at least five genes are compared.

43. A method of claim 40, wherein the expression levels of at least ten genes are compared.

44. A method of claim 40, further comprising the step of displaying the level of expression of at least one gene in the tissue or cell sample compared to the expression level when exposed to a toxin.

45. A method of claim 9, wherein the known toxin is a renal toxin.

46. A method of claim 42, wherein the renal toxin is selected from the group consisting of indomethacin, diflunisal, colchicine, chloroform, diclofenac, menadione, sodium chromate, sodium oxalate, thioacetamide and vancomycin.

47. A method of any one of claims 6-10, wherein nearly all of the genes in Tables 1-5 are detected.

48. A method of claim 47, wherein all of the genes in at least one of Tables 5-5L are detected.

49. A kit comprising at least one solid support of any one of claims 29-33 packaged with gene expression information for said genes.

50. A kit of claim 49, wherein the gene expression information comprises gene expression levels in a tissue or cell sample exposed to a renal toxin.

51. A kit of claim 50, wherein the gene expression information is in an electronic format.

52. A method of any one of claims 6-10, wherein the compound exposure is in vivo or in vitro.

53. A method of any one of claims 6-10, wherein the level of expression is detected by an amplification or hybridization assay.

54. A method of claim 53, wherein the amplification assay is quantitative or semi-quantitative PCR.

55. A method of claim 53, wherein the hybridization assay is selected from the group consisting of Northern blot, dot or slot blot, nuclease protection and microarray assays.

56. A method of identifying an agent that modulates at least one activity of a protein encoded by a gene in Tables 1-5 comprising:
(a) exposing the protein to the agent; and
(b) assaying at least one activity of said protein.

57. A method of claim 56, wherein the agent is exposed to a cell expressing the protein.

58. A method of claim 57, wherein the cell is exposed to a known toxin.

59. A method of claim 58 wherein the toxin modulates the expression of the protein.

60. A computer system of claim 34, wherein the genes are rat genes.
